(19) United States
(12) Patent Application Publication
(10) Pub. No.: US 2006/0141518 A1
(43) Pub. Date: Jun. 29, 2006

(54) Detection of gene expression

(76) Inventors: Kai Q. Lao, Pleasanton, CA (US); Mark Reed, Menlo Park, CA (US)

Correspondence Address: HARNESS, DICKEY & PIERCE, P.L.C., P.O. BOX 828, BLOOMFIELD HILLS, MI, 48303, US

(21) Appl. No.: 11/320,440

(22) Filed: Dec. 28, 2005

Related U.S. Application Data
(63) Continuation-in-part of application No. 11/090,468, filed on Mar. 24, 2005.
(60) Provisional application No. 60/639,765, filed on Dec. 28, 2004.
(60) Provisional application No. 60/556,157, filed on Mar. 24, 2004.
(60) Provisional application No. 60/556,224, filed on Mar. 24, 2004.
(60) Provisional application No. 60/556,162, filed on Mar. 24, 2004.
(60) Provisional application No. 60/556,163, filed on Mar. 24, 2004.
(60) Provisional application No. 60/630,681, filed on Nov. 24, 2004.

(51) Int. Cl. C12Q 1/68; C12P 19/34

(52) U.S. Cl. 435/6; 435/91.2

(57) ABSTRACT

Methods for detection of nucleic acids such as a cDNA copy of an mRNA are disclosed. The methods comprise using a PCR to form a preamplification product which comprises cDNA sequence as well as primer target sequences and a detection probe sequence, which are introduced by the forward and reverse primers. In a second PCR, preamplification product is amplified using universal primers which hybridize to the primer target sequences or their complements. Amplification can be detected using a detection probe that hybridizes to the detection probe sequence or its complement.

### CROSS-REFERENCE TO RELATED APPLICATIONS

This application claims priority from U.S. Provisional Application No. 60/639,765 to Lao et al., filed on Dec. 28, 2004; and this application is a Continuation-in-Part of copending U.S. patent application Ser. No. 11/090,468 to Lao et al., filed on Mar. 24, 2005, which claims priority from U.S. Provisional Application No. 60/556,157 to Chen et al., U.S. Provisional Application No. 60/556,224 to Andersen et al., U.S. Provisional Application No. 60/556,162 to Livak et al., and U.S. Provisional Application No. 60/556,163 to Lao et al., all filed on Mar. 24, 2004, and from U.S. Provisional Application No. 60/630,681 to Chen et al., filed on Nov. 24, 2004. The disclosures of the above applications are incorporated herein by reference.

### REFERENCE TO A SEQUENCE LISTING

The Sequence Listing, submitted Dec. 28, 2005 on compact disc, in two copies, Copy 1 and Copy 2, each containing the file named “Sequence Listing 9692-000052 (ST25).txt,” created Dec. 28, 2005, of 70 KB size, is hereby incorporated by reference.

### INTRODUCTION

Currently, genomic analysis, including that of the estimated 30,000 human genes is a major focus of basic and applied biochemical and pharmaceutical research. Such analysis may aid in developing diagnostics, medicines, and therapies for a wide variety of disorders. However, the complexity of the human genome and the interrelated functions of genes often make this task difficult. There is a continuing need for methods and apparatus to aid in such analysis.

### BRIEF DESCRIPTION OF THE DRAWINGS

The skilled artisan will understand that the drawings described herein are for illustration purposes only. The drawings are not intended to limit the scope of the present teachings in any way:

FIG. 1 illustrates an initial mixture for formation of a preamplification product, the mixture comprising a target nucleic acid, a forward primer, and a reverse primer;

FIG. 2 illustrates a detection mixture comprising a preamplification product, a first universal primer, a second universal primer, and a detection probe;

FIG. 3 illustrates a detection mixture for detecting a plurality of preamplification products, the mixture comprising different detection probe sequences and the detection probes comprise different fluorophores;

FIG. 4 illustrates a detection mixture comprising a first probe, which hybridizes to the complement of a detection probe sequence, and a second probe, which hybridizes to the target nucleic acid (or its complement);

FIG. 5 illustrates an initial mixture for detecting a cDNA, the mixture comprising an exon-exon junction;

FIG. 6 illustrates an initial mixture for formation of a preamplification product, the mixture comprising a target nucleic acid, a forward primer and a shorter reverse primer;

FIG. 7 illustrates an initial mixture for formation of a multiplex preamplification product, the mixture comprising multiple forward primers and multiple reverse primers.

### DESCRIPTION OF SOME EMBODIMENTS

The following description of some embodiments is merely exemplary in nature and is in no way intended to limit the present teachings, applications, or uses. Although the present teachings will be discussed in some embodiments as relating to polynucleotide amplification, such as PCR, such discussion should not be regarded as limiting the present teachings to only such applications.

All literature and similar materials cited in this application, including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including, but not limited to, defined terms, term usage, described techniques, or the like, this application controls.

The present teachings disclose methods for detecting a target nucleic acid, such as, for example, a cDNA of an mRNA. The methods, which utilize two-stage PCR assays, can provide quantitative data regarding the amount of a target nucleic acid present in a sample. The methods can utilize as forward or reverse primers, or both, in the first stage, at least one target nucleic acid-specific primer that adds, to the target nucleic acid(s), both (i) a preselected first primer target sequence for use in the second stage and (ii) a preselected selection probe sequence. Where only the forward primers or only the reverse primers provide both of the sequences (i) and (ii), the remaining member(s) of the primer pair(s) are target sequence-specific primers that can optionally add (iii) a preselected second primer target sequence, different from or the same as the first primer target sequence (i).

A preselected primer target sequence (i) and/or (iii) can be universal in that the same primer target sequence is added to all of the various target nucleic acids, present in a sample, that are chosen for detection in the assay. Selecting such a universal sequence as the first primer target sequence (i) can help eliminate bias that may be present in PCR replication; selecting such a universal sequence as the second primer sequence (iii) can further help eliminate such bias. Selection of such universal sequences can also decrease the number of different primers that would otherwise need to be synthesized for use in the second phase of the assay.

A preselected selection probe sequence can be universal in that the same selection probe sequence can be used in one primer that is present in each of a plurality of assays of different samples, wherein that one primer in each such assay can be specific for a different target nucleic acid. Note that, as used herein, the term plurality indicates at least two. Thus, the number of different selection probes that would otherwise need to be synthesized can be greatly decreased, since copies of the same selection probe useful in one assay are equally useful in another assay. In the case of multiplex assays, this can permit the same set of selection probes to be used in a variety of different assays. By way of illustration, in some embodiments, a series of 20-plex assays, in each of which 20 different target nucleic acids are assayed, can utilize the same set of 20 selection probes, regardless of the number of series members, so that if 2, 5, 10, 20, or more assays are being performed, then respectively 40, 100, 200, 400, or more different target nucleic acids can be simultaneously assayed using only 20 selection probes. Where the selection probes used in a multiplex assay further comprise a detectable label or labels, each of the labels can be unique to the selection probe of which it is a part, although the same label can be used in different assays, or in different loci of a given assay, as part of the same selection probe.

These methods can be used in a variety of applications, such as, for example, in clinical diagnosis of disease and in laboratory research studies. Some embodiments can be applied to the analysis of genomic DNA. Certain basic principles of PCR are set forth in U.S. Pat. Nos. 4,683,195, 4,683,202, 4,800,159, and 9,965,188, each issued to Mullis et al. Some embodiments of the present teachings can provide real-time PCR as described, for example, in PCT Publication No. WO 95/301139 and the progeny of U.S. patent application Ser. No. 08/235,411, such as U.S. Pat. Nos. 5,928,907 and 6,015,674.

In some embodiments, a target nucleic acid that can be detected using methods disclosed herein can be single-stranded DNA or a double-stranded DNA. In some embodiments, the strand or strands of a target nucleic acid can comprise at least about 10 nucleotides, at least about 20 nucleotides, at least about 50 nucleotides, at least about 100 nucleotides, or at least about 500 nucleotides. Except where noted otherwise, descriptions of nucleic acids herein refer to single-stranded DNA molecules, although the present disclosure is not limited to such molecules. In some embodiments, double-stranded molecules can be denatured to form single stranded molecules using denaturation methods well-known to skilled artisans. In some embodiments, the target nucleic acid can be single-stranded RNA, e.g., mRNA, wherein the polymerase is an RNA-dependent DNA polymerase, or “reverse transcriptase,” and a primer used to prime the reverse transcription reaction is specific for a target RNA.

In some embodiments, methods of detecting a target nucleic acid, such as, for example, a cDNA of an mRNA, can comprise forming an initial mixture by combining: (a) a sample suspected of comprising the target nucleic acid; (b) a polymerase; (c) a forward primer comprising a 5′ portion comprising a first primer target sequence and a 3′ portion that hybridizes to the target nucleic acid; (d) a reverse primer comprising a 5′ portion comprising a second primer target sequence and a 3′ portion that hybridizes to a complement of the target nucleic acid, and (e) a PCR reaction mix; followed by placing the resulting combination under conditions in which at least the forward primer is elongated, using the target nucleic acid as a template.

In some embodiments, an initial mixture can be formed under conditions in which at least the forward primer elongates, using the target nucleic acid as template. As used herein, “elongates” and “elongation” refer to the addition of one or more nucleotides to the 3′ end of a strand of a nucleic acid. If the target nucleic acid is a single-stranded DNA such as, for example, a single stranded cDNA, the forward primer can hybridize with the target nucleic acid. Hybridization between two nucleobase sequences, as used herein, can refer to hybridization between two complementary sequences, each comprising at least about 10 contiguous nucleotides, but does not imply that either nucleobase sequence is necessarily hybridized along its entire length. As used herein, “hybridization” refers to hybridization under high stringency conditions and such conditions are well-known to skilled artisans. In some embodiments, a polymerase used in the methods described herein can be a DNA polymerase. A “DNA polymerase” as used herein can be a thermostable DNA polymerase, such as, for example, a Taq polymerase. Furthermore, a DNA polymerase can have, in some embodiments, 5′ exonuclease activity.

In some embodiments, a forward primer hybridized to a target nucleic acid can be elongated using a DNA polymerase, with the target nucleic acid acting as template. If the target nucleic acid is a double-stranded DNA, such as, for example, a cDNA subjected to ‘second strand’ synthesis, the double stranded molecule can be denatured, for example, by thermal denaturation. The denatured strands can then be annealed to the forward and reverse primers under hybridization-promoting conditions such as, for example, a hybridization-promoting temperature. Denaturation, in some embodiments, can comprise chemical denaturation or thermal denaturation, for example denaturation by heating of a sample to about 90° C., to about 92° C., to about 95° C., to about 97° C., or to about 100° C., and such denaturation methods are well-known to skilled artisans.

In some embodiments, a forward primer can comprise a 5′ portion comprising a first primer target sequence and a 3′ portion that hybridizes to the target nucleic acid, and a reverse primer can comprise a 5′ portion comprising a second primer target sequence and a 3′ portion that hybridizes to a complement of the target nucleic acid. The forward primer and the reverse primer can each comprise at least about 20 nucleotides, at least about 30 nucleotides, or at least about 50 nucleotides, and up to about 60 nucleotides or up to about 70 nucleotides. Furthermore, in some embodiments, each portion of a primer comprising a primer target sequence can comprise at least about 10 nucleotides, at least about 15 nucleotides, at least about 20 nucleotides, at least about 25 nucleotides, or at least about 30 nucleotides, and up to about 40 nucleotides, up to about 50 nucleotides, up to about 60 nucleotides, or up to about 70 nucleotides, and each portion of a primer that hybridizes with a target nucleic acid or its complement can comprise at least about 10 nucleotides, at least about 15 nucleotides, at least about 20 nucleotides, at least about 25 nucleotides, or at least about 30 nucleotides, and up to about 40 nucleotides, up to about 50 nucleotides, up to about 60 nucleotides, or up to about 70 nucleotides. In regard to forward and reverse primers, as used herein, the portion of a forward primer that hybridizes to a target nucleic acid can hybridize at a position on the target nucleic acid situated 3′ to the position on the target nucleic at which a complement of the reverse primer can hybridize.

In some embodiments, an assay can include a detection mixture and can be a homogenous polynucleotide amplification assay, for coupled amplification and detection, wherein the process of amplification generates a detectable signal and the need for subsequent sample handling and manipulation to detect the amplified product is minimized or eliminated. Homogenous assays can provide for amplification that is detectable without opening a sealed well (or detection chamber) or further processing steps once amplification is initiated. Such homogenous assays can be suitable for use in conjunction with detection probes. For example, in some embodiments, the use of a detection probe, specific for detecting a particular target sequence, can be included in an amplification reaction in addition to a forward primer and a reverse primer of the present teachings. Homogenous assays among those useful herein are described, for example, in commonly assigned U.S. Pat. No. 6,814,934.

In some embodiments, at least one of the forward primer and the reverse primer can further comprise a detection probe sequence, which is a preselected nucleotide sequence to which a detection probe can hybridize. A detection probe sequence (or its complement) can be situated within the forward primer between the first target primer sequence and the sequence complementary to the target nucleic acid or within the reverse primer between the second primer target sequence and the sequence complementary to the target nucleic acid. A detection probe sequence can comprise at least about 10 nucleotides, at least about 15 nucleotides, at least about 20 nucleotides or at least about 30 nucleotides, up to about 50 nucleotides, up to about 60 nucleotides, or up to about 70 nucleotides. A detection probe sequence (or its complement), can be, in some embodiments, a “Zip-Code™” sequence commercially available from Applied Biosystems, Foster City, Calif.

In some embodiments, hybridization of a primer and a target DNA can comprise subjecting the initial mixture to annealing conditions such as, for example, thermal conditions which support annealing. Thermal conditions for annealing can include, for example, a hybridization-promoting temperature of from about 37° C. to about 72° C. or from about 37° C. to about 65° C. In some embodiments, the annealing temperature can be about 50° C., about 55° C., about 60° C., about 65° C., or about 70° C. In some embodiments, the initial elongation of the forward primer can occur while the initial mixture is incubated at a hybridization promoting temperature.

In some embodiments, conditions supporting elongation of a primer hybridized to a target nucleic acid in an initial mixture can comprise incubating the mixture at an elongation-promoting temperature such as, for example, from about 55° C. to about 72° C. The duration of each of these temperature conditions can be, for example, about 30 seconds, about 60 seconds, or about 90 seconds. Various other conditions such as, for example, inclusion of dNTPs in a mixture, can be in accordance with principles and methodologies of PCR well-known to skilled artisans.

In some embodiments, an initial mixture can be subjected to at least one cycle of a PCR, comprising thermal denaturation, annealing, and extension, whereby a preamplification product can be formed. This PCR can comprise two or more cycles and, for example, about 10 cycles of denaturation, annealing and elongation. In some embodiments, this polymeric chain reaction can comprise more than 10 cycles. In some embodiments, the preamplification is within the linear phase of amplification and as the reaction progresses, some of the reagents are being consumed as a result of amplification. The reactions start to slow down and the PCR product is no longer being doubled at each cycle. This linear amplification can be seen in the linear phase of the reaction. In some embodiments, the preamplification product is formed at temperatures lower than the temperature needed to form amplification products. For example, elongation during preamplification can be at a temperature of about 60° C. and elongation during amplification can be at a temperature of about 72° C. The use of two different temperatures for elongation for amplification and preamplification is advantageous because the primers for both steps can be in the same assay with minimal to no amplification of one of the sets of primers at one of the temperatures. In some embodiments, a preamplification product can comprise a double-stranded DNA molecule, in which one strand comprises, in 5′ to 3′ order, a forward primer sequence, a sequence complementary to the target nucleic acid, and a sequence complementary to the reverse primer. A preamplification product can further comprise a detection probe sequence.

In some embodiments, a detection mixture can be formed which can comprise a preamplification product, a first universal primer which hybridizes to a complement of the first primer target sequence, and a second universal primer which hybridizes to a complement of the second primer target sequence. In some embodiments, a first primer target sequence and a second primer target sequence can be identical. In some embodiments, the universal primers can also be identical, i.e. a detection mixture need comprise only one universal primer. Any preamplification product comprised by the detection mixture can be amplified by a PCR. In some embodiments, the PCR can comprise at least about 10 cycles of denaturation, annealing and elongation, up to about 20 cycles, up to about 25 cycles, up to about 30 cycles, up to about 35 cycles, or greater number of cycles. Denaturation can comprise chemical denaturation or thermal denaturation, for example as described supra. For annealing between a primer and a target DNA, the annealing temperature can be, for example, between about 37° C. to about 72° C., or from between about 65° C. to about 72° C. The conditions supporting elongation can comprise further including in the mixture dNTPs and incubating the mixture at an elongation-promoting temperature, such as, for example from about 65° C. to about 72° C. The duration of each of these temperature conditions can be, for example, about 30 seconds, about 60 seconds, or about 90 seconds.

In some embodiments, primer sequences can be selected using well-known principles for predicting melting temperature (for example, see, Owczarzy et al., Biopolymers, 44:217-239 (1998)). In some embodiments, melting temperatures for a hybridization of a forward primer and a target nucleic acid, or a reverse primer and a target nucleic acid, can be lower than the melting temperatures for hybridizations between the universal primers and the forward or reverse primer hybridized sequences. In non-limiting example, the melting temperature for a hybridization of a forward primer and a target nucleic acid, or a reverse primer and a target nucleic acid, can be about 65° C., and the melting temperature for a hybridization of a universal primer and a primer hybridized sequence can be about 72° C. In some embodiments, PCR amplification of the initial mixture can be conducted using a reannealing temperature of about 65° C., and PCR amplification of the detection mixture can be conducted using a reannealing temperature of about 72° C. In using such different PCR amplification reannealing temperature, it can be expected that non-elongated forward and reverse primers can be comprised by a detection mixture without significantly interfering with amplification of a preamplification product.

Detection of amplification of any preamplification product can reveal the presence and quantity of the target nucleic acid. In some embodiments, the amount of amplification detected can be proportional to the amount of preamplification product, which, in turn, can be proportional to the amount of the target nucleic acid. Hence, in some embodiments, quantification of amplification can be used to quantify the amount of the target nucleic acid. Detection of amplification can comprise, for example, detection of an amplified preamplification product using standard nucleic acid detection methods well-known to skilled artisans, such as, for example, electrophoretic methods, including gel electrophoresis or capillary electrophoresis.

In some embodiments, a detection mixture can further comprise a detection probe. A detection probe can comprise a sequence that hybridizes to the detection probe sequence or a complement thereof. The detection probe can be used to detect and to quantify the preamplification product. A detection probe, which can hybridize to a detection probe sequence, can comprise at least about 10 contiguous nucleobases, at least about 20 contiguous nucleobases, at least about 30 contiguous nucleobases, or at least about 40 contiguous nucleobases, up to about 50 contiguous nucleobases, and up to about 60 contiguous nucleobases or up to about 70 contiguous nucleobases.

Accordingly, in some embodiments, detection of amplification can comprise detection of hybridization of a detection probe to a detection probe sequence, as discussed below. As used herein, “detecting amplification” and “detection of amplification” can refer to detecting the quantity of an amplification product, or detecting accumulation of a product of an enzyme-catalyzed reaction which is coupled with a PCR. Such a product of an enzyme-catalyzed reaction can be, for example, an unquenched fluorophore that accumulates as a result of 5′-nuclease activity of a DNA polymerase that during a PCR, hydrolyzes a probe comprising a fluorophore, a nucleotide sequence, and a fluorescence quencher such as, for example, a TaqMan® probe (commercially available from Applied Biosystems, Foster City, Calif.). In some embodiments, detection of amplification can comprise detecting accumulation of an unquenched fluorophore such as, for example, the fluorophore of a fluorophore-comprising probe that is initially quenched prior to hybridization, but becomes unquenched upon hybridization to a detection probe sequence.

In some embodiments, a detection probe comprises a moiety that facilitates detection of a nucleic acid sequence, and in some embodiments, quantitative detection. In some embodiments, a detection probe can comprise, for example, a fluorophore such as a fluorescent dye, a fluorescent nanoparticle, a biotin, a hapten such as digoxygenin, a radioisotope, an enzyme, or an electrophoretic mobility modifier. In some embodiments, the level of amplification can be determined using a fluorescently labeled oligonucleotide. In some embodiments, a detection probe can comprise a fluorophore and a fluorescence quencher.

In some embodiments, a detection probe can comprise a fluorophore and can be, for example: a 5′-exonuclease assay probe such as a TaqMan® probe; a stem-loop Molecular Beacon described, for example, in U.S. Pat. Nos. 6,103,476 and 5,925,517, Tyagi et al., Nature Biotechnology 14:303-308 (1996); Vet et al., Proc. Nat'l Acad. Sci. USA 96:6394-6399 (1999); a stemless or linear molecular beacon described, for example, in PCT Patent Publication No. WO 99/21881; a Peptide Nucleic Acid (PNA) Molecular Beacon™ described, for example, in U.S. Pat. Nos. 6,355,421 and 6,593,091; a linear PNA Molecular Beacon described, for example, in Kubista et al., SPIE 4264:53-58 (2001); a flap endonuclease probe described, for example, in U.S. Pat. No. 6,150,097; a Sunrise®/Amplifluor® probe described, for example, in U.S. Pat. No. 6,548,250; a stem-loop and duplex Scorpion™ probe described, for example, in Solinas et al., Nucleic Acids Research 29:E96 (2001) and U.S. Pat. No. 6,589,743; a bulge loop probe described, for example, in U.S. Pat. No. 6,590,091; a pseudo-knot probe described, for example, in U.S. Pat. No. 6,589,250; a cyclicon described, for example, in U.S. Pat. No. 6,383,752; an MGB Eclipse™ probe (commercially available from Epoch Biosciences, Bothell, Wash.); a hairpin probe described, for example, in U.S. Pat. No. 6,596,490; a peptide nucleic acid (PNA) light-up probe; a self-assembled nanoparticle probe; or a ferrocene-modified probe described, for example, in U.S. Pat. No. 6,485,901; Mhlanga et al., Methods 25:463-471 (2001); Whitcombe et al., Nature Biotechnology 17:804-807 (1999); Isacsson et al., Molecular Cell Probes 14:321-328 (2000); Svanvik et al., Anal. Biochem. 281:26-35 (2000); Wolffs et al., Biotechniques 766:769-771 (2001), Tsourkas et al., Nucleic Acids Research 30:4208-4215 (2002); Riccelli et al., Nucleic Acids Research 30:4088-4093 (2002); Zhang et al., Sheng Wu Hua Xue Yu Sheng Wu Li Xue Bao (Shanghai) (Acta Biochimica et Biophysica Sinica) 34:329-332 (2002); Maxwell et al., J. Am. Chem. Soc. 124:9606-9612 (2002); Broude et al., Trends Biotechnol. 20:249-56 (2002); Huang et al., Chem. Res. Toxicol. 15:118-126 (2002); Yu et al., J. Am. Chem. Soc. 14:11155-11161 (2001).

In some embodiments, a detection probe can comprise a sulfonate derivative of a fluorescent dye, a phosphoramidite form of fluorescein, or a phosphoramidite form of CY5. Further detection probes useful herein include those described, for example, in U.S. Pat. Nos. 5,188,934, 5,750,409, 5,847,162, 5,853,992, 5,936,087, 5,986,086, 6,020,481, 6,008,379, 6,130,101, 6,140,494, 6,140,500, 6,191,278, and 6,221,604. Further energy transfer dyes useful herein include those described in U.S. Pat. Nos. 5,728,528, 5,800,996, 5,863,727, 5,945,526, 6,335,440, and 6,849,745, U.S. patent application Publication No. 2004/0126763 A1, PCT Publication No. WO 00/13026A1, PCT Publication No. WO 01/19841A1, U.S. Patent Application Ser. No. 60/611,119, filed Sep. 16, 2004, and U.S. patent application Ser. No. 10/788,836, filed Feb. 26, 2004. In some embodiments, a detection probe can comprise a fluorescence quencher such as any one of a black hole quencher (commercially available from Metabion International AG, Germany), an Iowa Black™ quencher (commercially available from Integrated DNA Technologies, Coralville, Iowa), a QSY quencher (commercially available from Molecular Probes, Carlsbad, Calif.), as well as Dabsyl and Eclipse™ Dark Quenchers (commercially available from Epoch Biosciences, Inc., Bothell, Wash.).

In some embodiments, a detection probe can comprise a fluorescent dye. In some embodiments, the fluorescent dye can comprise at least one of rhodamine green (R110), 5-carboxyrhodamine, 6-carboxyrhodamine, N,N′-diethyl-2′,7′-dimethyl-5-carboxy-rhodamine (5-R6G), N,N′-diethyl-2′,7′-dimethyl-6-carboxyrhodamine (6-R6G), 5-carboxy-2′,4′,5′,7′,−4,7′-hexachlorofluorescein, 6-carboxy-2′,4′,5′,7′,4,7-hexachloro-fluorescein, 5-carboxy-2′,7′-4′,5′-dichlorofluorescein, 6-carboxy-2′,7′-dicarboxy-4′,5′-dichlorofluorescein, 5-carboxy-2′,4′,5′,7′-tetrachlorofluorescein, 1′,2′-benzo-4′-fluoro-7′,4,7-trichloro-5-carboxyfluorescein, 1′,2′-benzo-4′-fluoro-7′,4,7-trichloro-6-carboxy-fluorescein, or 1′,2′,7′,8′-dibenzo-4,7-dichloro-5-carboxyfluorescein.

In some embodiments, amplified sequences can be detected in double-stranded form by a detection probe comprising an intercalating or a crosslinking dye, such as ethidium bromide, acridine orange, or an oxazole derivative, for example, SYBR Green® (commercially available from Molecular Probes, Carlsbad, Calif.), which exhibits a fluorescence increase or decrease upon binding to double-stranded nucleic acids. In some embodiments, a detection probe comprises SYBR Green® or Pico Green® (commercially available from Molecular Probes, Carlsbad, Calif.).

In some embodiments, a detection probe can comprise, as fluorophore, at least one fluorescent nanoparticle, such as a fluorescent semiconductor nanoparticle, which can comprise, e.g.: CdTe, CdSe, CdS, ZnSe, ZnTe, CdSSe, CdZnTe, or ZnCdS; or a core comprising such a material within at least one shell that can comprise, e.g., ZnS or CdSe. In some embodiments, the fluorescent nanoparticles can comprise at least one quantum dot. Quantum dots are available from Quantum Dot Corp., Hayward, Calif., USA, and they can be formed, made aqueous-compatible, and linked to nucleic acids or nucleic acid analogs for use in assays, by methods described, e.g., in: U.S. Pat. Nos. 5,990,479; 6,251,303; 6,274,323; 6,322,901; 6,426,513; 6,500,622; 6,576,291; 6,649,138; 6,815,064; and the patent family members thereof. As is true for other detectable labels useful herein, quantum dots can be pre-treated by attachment to a binding partner to form quantum dot conjugates, such as QDOT-biotin, QDOT-avidin, QDOT-maleimide, QDOT-carboxyl, or QDOT-(amino-functionalized) polyethylene glycol conjugates, which are also available from Quantum Dot Corp.

In some embodiments, a detection probe can comprise an enzyme that can be detected using an enzyme activity assay. An enzyme activity assay can utilize a chromogenic substrate, a fluorogenic substrate, or a chemiluminescent substrate. In some embodiments, the enzyme can be an alkaline phosphatase, and the chemiluminescent substrate can be (4-methoxyspiro [1,2-dioxetane-3,2′(5′-chloro)-tricyclo [3.3.1.13, 7]decan]-4-yl) phenylphosphate. In some embodiments, a chemiluminescent alkaline phosphatase substrate can be CDP-Star® chemiluminescent substrate or CSPD® chemiluminescent substrate (commercially available from Applied Biosystems, Foster City, Cailf., USA).

In some embodiments, a detection probe label can be a radioisotope, such as, for example, a moiety comprising ³H, ¹⁴C, ³⁵S, ³²P, ³³P, or ¹²¹I. The radioisotope can be detected using well-known methods, such as autoradiography or scintillation counting. In some embodiments, a detection probe label can be a luminescent lanthanide complex, such as a lanthanide chelate, which comprises a trivalent lanthanide cation of any lanthanide isotope.

In some embodiments, a detection probe comprising a fluorophore can further comprise a fluorescence quencher. A fluorescence quencher can be any fluorescence quencher well-known to skilled artisans, such as a fluorescent fluorescence quencher such as TAMRA, or a non-fluorescent fluorescence quencher such as a combined non-fluorescent quencher-minor groove binder. A detection probe, in some embodiments, can be used in various fluorogenic assays in which a fluorophore comprised by a probe is initially quenched. A fluorogenic assay utilizing a fluorescence quencher can be, for example, a 5′-nuclease assay such as for example, a TaqMan® assay commercially available from Applied Biosystems, Foster City, Calif., USA. In these embodiments, the 5′-nuclease assay can utilize the 5′ nucleolytic activity of a DNA polymerase that catalyzes a PCR amplification of a probe-hybridized template, such as a probe set ligation sequence.

In some embodiments, a fluorogenic detection assay can be a real-time PCR assay or an end-point PCR assay. Using a fluorogenic detection assay, quantitative results can be obtained, for example, with the aid of a fluorimeter, such as a fluorimeter comprised by an integrated nucleic acid analysis system. In a non-limiting example, an integrated nucleic acid analysis system that can be used to practice methods disclosed herein can be an Applied Biosystems ABI PRISM® 7900HT Sequence Detection System.

In some embodiments, a detection probe can comprise an electrophoretic mobility modifier. As used herein, a “mobility modifier” can be a nucleobase polymer sequence that can increase the size of a detection probe, or can be a non-nucleobase moiety that increases drag coefficient of the detection probe. Examples of a mobility modifier among those useful herein can include those described in U.S. Pat. Nos. 5,470,705, 5,514,543, 5,580,732, and 5,624,800. A detection probe comprising a mobility modifier can exhibit a relative mobility in an electrophoretic or chromatographic separation medium that allows a user to identify and distinguish the detection probe from other molecules comprised by a sample. In some embodiments, a detection probe comprising a sequence complementary to a detection probe sequence and an electrophoretic mobility modifier can be, for example, a ZipChute™ probe commercially by available from Applied Biosystems, Foster City, Calif., USA. In some embodiments, at least one of a target sequence, a preamplification product, an amplification product, or a detection probe can be immobilized on a solid support. In these embodiments, hybridization of a detection probe with an amplification product, followed by electrophoretic analysis, can be used to determine the identity and quantity of a target nucleic acid.

In some embodiments, at least one of the first and the second detection probes can further comprise an electrophoretic mobility modifier. Moreover, in some embodiments, detecting amplification of a preamplification product can comprise detection by an electrophoresis assay such as a capillary electrophoresis assay or a gel electrophoresis assay.

In various embodiments employing detection of colored, fluorescent, or luminescent signals emitted from detectable labels, a plurality of different signals can be detected in a single locus, e.g., a single well or single spot. This type of multiple-detection can be performed at each locus on a multi-locus assay format, such as a multi-locus card or plate. This permits detection of a plurality of different target nucleic acids in a single locus in some embodiments of a multiplex assay. In such embodiments, each such signal in a given locus can be unique to one of the target nucleic acids being assayed at the locus. This can be accomplished by selecting, as a detectable label attached to the unique probe for each target nucleic acid to be assayed at a given locus, a label that can produce a signal unique to that probe. Thus, a multiplex assay hereof can be performed to detect two or more target nucleic acids per locus. In some embodiments, the set of detectable labels selected for use in such a locus can be up to about 4, up to about 8, or more. Where fluorescent labels are used, in some embodiments, up to 8 different labels can be selected; or up to about 4 different labels; yet, in the case of, e.g., fluorescent semiconductor nanoparticles or any narrow-wavelength-range detectable labels, such as those having a half-maximum peak width of about 30 nm or less, even more than 8 different labels can be used per locus. Other detection modes described herein can alternatively be employed to assay a plurality of different target nucleic acids from a single locus.

Therefore, in some embodiments, a multiplex assay can be performed in a single locus. In some embodiments, a multiplex assay can be performed by distributing, from a preamplification locus into separate loci, portions of a preamplified population of nucleic acids in which preamplified members thereof comprise at least one universal primer target sequence and at least one selection probe sequence. One or more than one target nucleic acid can then be assayed for detection in each separate locus, such as by performing a detection amplification reaction at each separate locus. Thus, in some embodiments, a multiplex assay can be performed by assaying one target nucleic acid in each of a plurality of separate loci; and in some embodiments, a multiplex assay can be performed by assaying multiple target nucleic acids in each of a plurality of separate loci. Multiplex assays hereof include all such assay formats that are capable of detecting more than one target nucleic acid per assay, whether, e.g.: single-multiplex-locus assays that can detect multiple target nucleic acids at a single locus; multiple-simplex-locus assays that can detect one target nucleic acid at each of a plurality of loci; multiple-multiplex-locus assays that can detect multiple target nucleic acids at each of a plurality of loci; or a combination thereof.

In some embodiments, assays can be performed in which each target nucleic acid is a single nucleotide sequence, although it is also possible to perform assays in which the target nucleic acid is a nucleic acid target that comprises a set of related nucleotide sequences, i.e. different molecules. For example, a nucleic acid target can comprise a set of sequences, i.e. different molecules, that all encode expression products having the same type of enzymatic activity, or that all share a particular sequence motif. In such a format, the assay can be designed to detect the selected set of sequences together: by selecting primers, used in forming preamplification products from the different members of the set, that contain the same detection probe sequence; or by selecting detection probes, for set-member preamplification products containing different detection probe sequences, that contain the same detectable label; or by using both the same detection probe sequence and the same detectable label for all set members.

In some embodiments, multiplex assays can be performed in which each detection probe present in a given detection mixture, i.e. at a given locus, is unique to one of the target nucleic acids to be detected in that detection mixture. In some embodiments, a detection probe can be one that comprises a nucleotide sequence capable of hybridizing to the detection probe sequence incorporated into the target nucleic acid preamplification product. In some embodiments, more than one detection probe, unique to the same target nucleic acid, can be used, wherein each of the “same-target” detection probes is specific for a different site on the target nucleic acid preamplification product, the different sites being exclusive of one another, i.e. non-overlapping. In each set of “same-target” detection probes, at least one of the probes will be specific for a detection probe sequence incorporated into the target nucleic acid preamplification product.

A detection probe unique for a given target nucleic acid to be detected in a given detection mixture can contain a detectable label; and the label can be unique to that detection probe. The same detection probe sequence can be incorporated into different preamplification products, wherein at least one of the primers to be used to amplify each such “same-detection-probe-sequence” preamplification product is unique to the target nucleic acid, rather than being a “universal” primer that is common to another nucleic acid present in the detection mixture; in such embodiments, each of the preamplification products that contains the same detection probe sequence can be amplified in a separate detection mixture. In each such separate detection mixture, the detection probe to be used to detect the “same-detection-probe-sequence” preamplification product member of that detection mixture, can contain the same label, or a different label as that used in another detection mixture for a different “same-detection-probe-sequence” preamplification product.

In some embodiments, different detection probe sequences, each unique to one of the target nucleic acids, can be incorporated into their preamplification products. The same label can be used to detect each of the preamplification products, wherein at least one of the primers to be used to amplify each such “same-label-detectable” preamplification product is unique to the target nucleic acid, rather than being a “universal” primer that is common to another nucleic acid present in the detection mixture; in such embodiments, each of the preamplification products that is to be detected using the same label can be amplified in a separate detection mixture, and the sequence of each labeled probe can be unique to one of the detection probe sequences. However, in some embodiments in which a different detection probe sequence is incorporated into each target nucleic acid preamplification product, different detection probes, each containing a different detectable label and each capable of hybridizing to only one of the detection probe sequences, can be used for detection in the same detection mixture, i.e. at the same locus. In some embodiments, the detection probe, and thus the incorporated detection probe sequence, or the label, or both, can be unique to one of the target nucleic acids to be detected in the entire assay, regardless of the number of detection mixtures or loci present in the assay.

In some embodiments, at least one of a target sequence or a detection mixture component can be attached to a solid support. In some embodiments, a detection mixture component such as, for example, a primer, a preamplification product or a detection probe, can comprise a binding moiety for which a binding partner is available. The association between a binding moiety and its binding partner can have, in a non-limiting example, a dissociation constant (Kd) smaller than about 10⁻⁶ M, smaller than about 10⁻⁷ M, smaller than about 10⁻⁸ M, smaller than about 10⁻¹⁰ M, or smaller than about 10⁻¹⁵ M. Non-limiting examples of a binding moiety and its binding partner include biotin and avidin, biotin and streptavidin, biotin and an anti-biotin antibody, and digoxygenin and an anti-digoxygenin antibody. In some embodiments, a solid support, including an attached binding partner, can be contacted with the detection mixture. Upon contact, the binding moiety can attach to its binding partner, and, as a consequence, become immobilized upon the solid support.

In some embodiments, a detection mixture component can be covalently attached to a solid support. Furthermore, in some embodiments, immobilization of a detection mixture component on a solid support can be reversible. In a non-limiting example, a detection probe comprising an electrophoretic mobility modifier (as discussed below) can be attached to a solid support using a reversible attachment. The probe can then be detected and identified, e.g., using electrophoresis, following its release from the solid support.

In some embodiments, at least one of the forward primer and the reverse primer can be attached to a solid support. In some embodiments, the attachment can be a covalent attachment, or a non-covalent attachment mediated by a binding moiety and binding partner attached to the solid support. Similarly, in some embodiments, a detection probe can be attached to a solid support, either covalently or non-covalently.

In some embodiments, a solid support can be, in a non-limiting example, a bead such as a polymer bead, a locus of a microcard, or a well of a microplate such as an ELISA or PCR plate.

In some embodiments, a microcard can be a device for detecting or quantitating one or more of a plurality of different analytes in a liquid sample. The microcard can include a substrate which defines a sample-distribution network having (i) a sample inlet, (ii) one or more detection chambers, and (iii) channel means providing a dead-end fluid connection between each of the chambers and the inlet. In some embodiments, each detection chamber includes an analyte-specific reagent effective to react with a selected analyte that can be present in the sample, and detection means for detecting the signal resulting from the reaction. In some embodiments, the detection means for each detection chamber includes an optically transparent window through which the signal can be detected optically. In some embodiment, the detection means includes a non-optical sensor for detecting the signal.

The channel means of the microcard can be configured in numerous ways. In some embodiments, the channel means can include a single channel to which the detection chambers are connected by dead-end fluid connections. In some embodiments, the channel means can include at least two different channels, each connected to a different group of detection chambers. In some embodiments, the channel means can include a single channel for each detection chamber.

In some embodiments, the microcard can include a vacuum port for placing the detection chambers under vacuum prior to the addition of a sample. The vacuum port can be connected to the channel means at a site between, and in fluid communication with, the sample inlet and the detection chambers. The vacuum port can be connected to the channel means at a site downstream of the detection chambers and the vacuum port can be additionally useful for removing liquid from the channel means after the detection chambers have been filled, to help isolate the detection chambers from one another and further reduce cross-contamination.

In some embodiments, the microcard includes means for regulating the temperatures of the detection chambers, and can provide temperature control between 0° C. and 100° C., for promoting the reaction of the sample with the detection reagents. In some embodiments, the temperature regulating means includes a conductive heating element for each detection chamber for rapidly heating the contents of the chamber to a selected temperature. The temperature control means can be adapted to regulate the temperatures of the detection chambers for heating and cooling the chambers in accordance with a selected assay protocol.

The microcard can be manufactured and packaged so that the sample-distribution network (e.g., sample inlet, detection chambers, and channel means) is provided under vacuum, ready for immediate use by the user. In some embodiments, the sample-distribution network is provided under atmospheric pressure, so that the evacuation step is carried out by the end-user prior to sample loading. The microcard also includes a substrate containing a plurality of sample-distribution networks, as described above, for testing a single sample or a plurality of samples for selected analytes.

In some embodiments, washing procedures can be used according to standard procedures to separate a component of a detection mixture attached to a solid support (directly or indirectly) from non-attached components. If the attachment is, for example, of a preamplification product attached to a locus of a microcard, the preamplification product can be amplified according to methods as discussed herein. An amplification product can be identified by virtue of its position in the microcard. Some embodiments involving a solid support can be multiplexed for analysis of multiple samples or multiple target nucleic acids.

In some embodiments, examples of the microcard among those useful herein include those described in commonly assigned U.S. Pat. Nos. 6,124,138 and 6,126,899.

In some embodiments, a microplate can be a device for detecting or quantitating one or more of a plurality of different analytes in a liquid sample. The microplate comprises a substantially planar substrate, having a first major surface and a second major surface. As referred to herein, a “substantially planar” surface is, or is capable of being, flat having substantially two-dimensional geometry (in x and y dimensions) considering the surface as a whole, although it can have surface irregularities in the third (z) dimension. A “major surface” of a substantially planar substrate refers to a surface that is defined by the x and y dimensions of the substrate.

The microplate substrate can have any dimension (in the x and y dimensions), but can be sized so as to readily handled during use, provide sufficient sample capacity, and be compatible with instrumentation used in amplification reactions. In some embodiments, the footprint dimensions of the microplate are essentially the standards as specified by the Society of Biomolecular Screening (SBS) and the American National Standards Institute (ANSI) standards, published January 2004 (ANSI/SBS 1-2004), incorporated by reference herein. In some embodiments, the footprint dimensions for the microplate are about 127.76 mm (5.0299 inches) in length and about 85.48 mm (3.3654 inches) in width. In some embodiments, the thickness of the plate can be from about 1.00 mm.

In some embodiments, the microplate comprises a plurality of wells. As referred to herein, a “well” is a feature formed in the substrate which is operable to contain a liquid during use of the microplate. Such wells have an opening, which is operable to allow the deposit of a liquid into the wells and the wells are formed in a major surface of the substrate. Examples of microplates among those useful herein include those described in U.S. patent application Ser. Nos. 11/086,261 and 11/096,282. In some embodiments, a solid support can be a substrate comprising oil wells and examples of such include those described in U.S. patent application Ser. No.10/944,686.

In some embodiments, the present teachings can employ any of a variety of universal detection approaches involving real-time PCR and related approaches. For example, the present teachings contemplate embodiments in which an encoding ligation reaction is performed in a first reaction vessel such as, for example, an Eppendorf tube®, and a plurality of decoding reactions are then performed on a solid substrate described herein. For example, a multiplexed oligonucleotide ligation reaction (OLA) can be performed to query a plurality of target DNA, wherein each of the resulting reaction products is encoded with, for example, a primer portion, and/or, a universal detection portion. By including a distinct primer pair in each of the plurality of wells of a microplate (or in the detection chambers of a microcard) corresponding to the primer sequences encoded in the OLA, a given encoded target DNA can be amplified by that distinct primer pair in a given well of the plurality of wells. Further, a universal detection probe such as, for example, a nuclease-cleavable TaqMan® probe, can be included in each of plurality of wells of microplate to provide for universal detection of a single universal detection probe. Such approaches can result in a universal microplate or a universal microcard, with its attendant benefits including, among other things, one or more of economies of scale, manufacturing, and/or ease-of-use. The nature of the multiplexed encoding reaction can comprise any of a variety of techniques, including a multiplexed encoding PCR pre-amplification or a multiplexed encoding OLA. Further, various approaches for encoding a first sample with a first universal detection probe, and a second sample with a second universal detection probe, thereby allowing for two sample comparisons in a single microplate or a single microcard, can also be performed according to the present teachings. Illustrative embodiments of such encoding and decoding methods can be found, for example, in PCT Publication No. WO2004/027081, PCT Publication No. WO2004/028342, and U.S. Provisional Application Nos. 60/556157, 60/556162, 60/556163, 60/556224, and 60/630681.

In some embodiments, a detection mixture can be formed that can comprise the preamplification product, a first universal primer that hybridizes to a complement of the first PCR primer target sequence, a second universal primer that hybridizes to a complement of the second PCR primer target sequence, a first detection probe comprising a sequence that hybridizes to the detection probe sequence or a complement thereof, a second detection probe comprising a sequence that hybridizes to portion of a sequence comprised by the target nucleic acid or a complement thereof, and a PCR reaction mix. Any preamplification product comprised by the detection mixture can be amplified by PCR. Detection of amplification of any preamplification product using the first and second detection probes can reveal the presence of the target nucleic acid. In some embodiments, the first and second detection probes can comprise different labels, for example, two different fluorophores.

In some embodiments, detection of amplification of any preamplification product can be quantitative detection, as described supra. Detection of amplification of any preamplification product can comprise quantifying the amplification detected by the first and second probes, and comparing the amounts of amplification revealed by each probe. In some embodiments, a quantitative signal from a first detection probe can be measured in a plurality of samples, and used, for example, as a standard for comparing samples.

In some embodiments, the present teachings set forth herein describe methods for detecting a plurality of target nucleic acids. The methods can comprise forming an initial mixture by combining component including: (a) a sample suspected of comprising the plurality of target nucleic acids; (b) a polymerase; (c) a plurality of primer sets, each primer set comprising: a forward primer comprising a 5′ portion comprising a first primer target sequence and a 3′ portion that hybridizes to a target nucleic acid, and a reverse primer comprising a 5′ portion comprising a second primer target sequence and a 3′ portion that hybridizes to a complement of the same target nucleic acid; and (d) a PCR reaction mix; wherein at least one of each forward primer and each reverse primer of a primer set further comprises a detection probe sequence unique for the primer set; and placing the resulting combination under conditions in which a forward primer elongates if hybridized to a target nucleic acid. The methods can further comprise forming a plurality of preamplification products by subjecting the initial mixture to at least one cycle of PCR, and forming a detection mixture comprising the plurality of preamplification products, a first universal primer that hybridizes to a complement of the first primer target sequence, a second universal primer that hybridizes to a complement of the second primer target sequence, and a PCR reaction mix; and amplifying any preamplification product comprised by the detection mixture. Each of the plurality of target nucleic acids can be detected by detecting amplification of any preamplification product.

In some embodiments, a detection mixture can further comprise a plurality of detection probes, wherein each detection probe comprises a sequence that hybridizes to a detection probe sequence comprised by a preamplification product or complement thereof. Because a detection probe sequence can be unique for each primer set, a plurality of detection probes, each comprising both a unique label and a sequence that hybridizes to a detection probe sequence, can be used to quantifiably measure a plurality of target nucleic acids comprised by a sample. Because high stringency hybridization conditions can be used, sequences of different detection probes can differ by as little as one nucleotide, two nucleotides, three nucleotides, or four nucleotides. The number of different target nucleic acids that can be detected in one sample can be as many as the number of distinguishable detection probe labels available. For example, detection probes in a single sample can include a variety of labels such as, for example: fluorescent and non-fluorescent labels; or a variety of different fluorophores having different excitation and/or emission spectra, such as a red light-emitting fluorophore, an orange light-emitting fluorophore, a yellow light-emitting fluorophore, a green light-emitting fluorophore, or a blue light-emitting fluorophore, as described above. In some embodiments, fluorescence can be stimulated using a radiation source which emits excitatory wavelengths of electromagnetic waves appropriate for particular fluorophores. For example, light from a laser or a mercury or a xenon light source filtered with appropriate optical filters, can be used to excite the fluorescence of a fluorophore comprised by a detection probe.

In some embodiments, the location of a fluorescent signal on a solid support such as, for example, a microcard or a microplate, can be indicative of the identity of a target nucleic acid comprised by a sample. In some embodiments, a plurality of detection probes can be distributed to identify loci of a solid support such as detection chambers of a microcard or wells of a microplate. In some embodiments, a plurality of preamplification products can be contacted with the loci of the solid support. The preamplification products comprised by the loci can be subjected to amplification conditions, in which each locus further comprises a first universal primer and a second universal primer. A signal deriving from a detection probe, such as, for example, an increase in fluorescence intensity of a fluorophore at a particular locus, can be detected if a preamplification product hybridizes to a detection probe and is then amplified. The location of the locus can indicate the identity of the target nucleic acid, and the intensity of the fluorescence can indicate the quantity of the target nucleic acid. In some embodiments, solid support such as, for example, a microcard or a microplate, can be used for multiplex target nucleic acid amplification detection assays.

In some embodiments, the present teachings describe methods for detecting a target nucleic acid in a plurality of samples. These methods can comprise forming a first initial mixture by combining components including (a) a first sample suspected of comprising the target nucleic acid, (b) a polymerase, (c) a forward primer comprising a 5′ portion comprising a first primer target sequence and a 3′ portion that hybridizes to the target nucleic acid, (d) a reverse primer comprising a 5′ portion comprising a second primer target sequence and a 3′ portion that hybridizes to a complement of the target nucleic acid, and (e) a PCR reaction mix, wherein at least one of the forward primer and the reverse primer further comprises a first detection probe sequence, and placing the resulting combination under conditions in which at least the forward primer elongates; forming a first preamplification product by subjecting the first initial mixture to at least one cycle of a PCR; forming a second initial mixture by combining components including (a) a second sample suspected of comprising the target nucleic acid, (b) a polymerase, (c) a forward primer comprising a 5′ portion comprising a first primer target sequence and a 3′ portion that hybridizes to the target nucleic acid, (d) a reverse primer comprising a 5′ portion comprising a second primer target sequence and a 3′ portion that hybridizes to a complement of the target nucleic acid, and (e) a PCR reaction mix, wherein at least one of the forward primer and the reverse primer further comprises a second detection probe sequence, and placing the resulting combination under conditions in which at least the forward primer elongates; and forming a second preamplification product by subjecting the second initial mixture to at least one cycle of a PCR. In some embodiments, the first and second detection probe sequences can be different sequences. The sequence can differ by as little as one nucleotide, two nucleotides, three nucleotides, four nucleotides, or greater. In some embodiments, a detection mixture can be formed which can comprise the preamplification products, a first universal primer which hybridizes to a complement of the first primer target sequence, and a second universal primer which hybridizes to a complement of the second primer target sequence. Any preamplification product comprised by the detection mixture can be amplified using a PCR; amplification of any preamplification product can be detected. In some embodiments, a detection mixture can further comprise a plurality of detection probes, each detection probe comprising a sequence which hybridizes to the detection probe sequence or a complement thereof comprised by a preamplification product.

Some embodiments of the present teachings include methods, reagents, compositions, and diagnostic kits, for use in simultaneously amplifying multiple nucleic acid targets. In particular, a two-step multiplex amplification reaction wherein the first step truncates a standard multiplex amplification round or cycle to thereby “boost” the sample copy number by only a 100-1000 or more fold, in terms of increase in the target nucleic acid copy number. Following the first step, the resulting product is divided into optimized secondary single amplification reactions, each containing one or more of the primer sets that were used previously in the first or multiplexed booster step. The booster step can be performed using an aqueous target nucleic acid or using a solid phase archived nucleic acid. An example of such a booster step among those useful herein can include those described in U.S. Pat. No. 6,605,451.

In some embodiments, the present teachings provide methods, reagents, and kits for carrying out a variety of assays suitable for analyzing polynucleotides or polynucleotide-containing samples, which methods can include a preamplification step performed in a multiplex fashion. Some embodiments provide methods for analyzing and improving the efficiency of amplification and for carrying out gene expression analysis or other analysis such as SNP, for example. Since a plurality of different sequences are preamplified simultaneously in a single reaction, the multiplex preamplifications can be used in a variety of contexts to effectively increase the concentration or quantity of a sample available for downstream analysis and/or assays. In some embodiments, downstream analysis and/or assays include methods such as, for example, PCR, real-time PCR, reverse transcriptase PCR, and the like. Once the sample has been multiplex-preamplified, it can be divided into aliquots, with or without prior dilution or-concentration, for subsequent analysis. Owing to its increased concentration or quantity, significantly more analysis or assays can be performed with multiplex-preamplified samples than could be performed with the original sample. In some embodiments, the multiplex preamplification even permits the ability to perform assays or analysis that would otherwise require more sample or a higher concentration of sample than was originally available; for example, after 1,000× multiplex preamplification, subsequent assays could then be performed with 1,000× less sample. In some embodiments, multiplex amplification allows the ability to perform downstream analysis for assays that may not have been possible with the original sample due to its limited quantity. Examples of multiplex preamplification techniques useful herein include those described in PCT Publication No. WO 2004/051218.

In some embodiments, a two-stage preamplification method can be used. The method preamplifies the sample in one vessel by IVT and, for example, this preamplification stage can be 100× sample in copy number. In the second stage, the preamplified product is divided into aliquots and preamplified by PCR, for example, and this preamplification stage can be 16,000× sample or more in copy number.

In some embodiments, the preamplification product is split into numerous portions and each portion can be quantitated using universal primers and a specific probe that is specific for a well-known correlation of a sequence. In some embodiments, the preamplification product can be split into wells of a microplate or detection chambers of a microcard. In some embodiments, the forming of a preamplification product can comprise subjecting the mixture to from one cycle up to about 10 cycles of PCR, or subjecting the mixture to from at least two cycles up to about 10 cycles of PCR.

In some embodiments, a detection mixture can be formed that can comprise both the first and the second preamplification products, a first universal primer that hybridizes to a complement of the first primer target sequence, and a second universal primer that hybridizes to a complement of the second primer target sequence. Any preamplification product comprised by the detection mixture can be amplified, and the amplification of any preamplification product can then be detected. In some embodiments, a detection mixture can further comprise a first detection probe comprising a sequence that hybridizes to the first detection probe sequence or a complement thereof and a second detection probe comprising a sequence that hybridizes to the second detection probe sequence or a complement thereof. The first and second detection probes can comprise different labels, for example, different fluorophores such as, for example, VIC and FAM. Sequences of the first and second detection probes can differ by as little as one nucleotide, two nucleotides, three nucleotides, four nucleotides, or greater, provided that hybridization occurs under conditions which allow each probe to hybridize specifically to its corresponding detection probe sequence.

In some embodiments, amplification can be detected in a single detection mixture, and the universal primers can be the same for amplification of both preamplification products; amplification conditions for the two samples can be similar, other than the probe labels and the detection probe sequences. Similar fluorescence intensity properties of the fluorophores can, in some embodiments, facilitate quantitative comparisons between two target nucleic acids comprised by a sample. In some embodiments, fluorophores can be selected such that their emission intensities, as detected by instrumentation, can be of similar magnitude for similar (unquenched) fluorophore concentrations, further facilitating quantitative comparisons between target nucleic acids comprised by a sample.

In some embodiments, a detection mixture can comprise the preamplification product, a universal primer that hybridizes to a complement of the first PCR primer target sequence, and the reverse primer. Any preamplification product comprised by the detection mixture can be amplified by PCR. Detection of amplification of any preamplification product can reveal the presence of the target nucleic acid. Furthermore, detection of amplification of any preamplification product can be quantitative detection. Because the amount of amplification detected can be proportional to the amount of preamplification product, which, in turn, can be proportional to the amount of the target nucleic acid, detection of amplification can be used to quantify the amount of the target nucleic acid originally present in a sample.

In some embodiments, the present teachings include methods for detecting a target nucleic acid such as a cDNA. The methods can comprise forming a mixture by combining components including (a) a sample suspected of comprising a target nucleic acid, (b) a polymerase, (c) a forward primer comprising a 5′ portion comprising a primer target sequence, a detection probe sequence portion, and a 3′ portion that hybridizes to the target nucleic acid, (d) a reverse primer that hybridizes to a complement of the target nucleic acid, and (e) a PCR reaction mix, and in some embodiments, by then placing the resulting combination under conditions in which at least the forward primer elongates, using the target nucleic acid as a template. By subjecting the mixture to at least one cycle of PCR, a preamplification product can form. In some embodiments, a preamplification product can comprise a double-stranded DNA molecule, in which one strand comprises a forward primer sequence and a sequence complementary to the target nucleic acid.

In some embodiments, a detection mixture can be formed, as described supra. A detection mixture can comprise the preamplification product, a universal primer which hybridizes to a complement of the first primer target sequence and the reverse primer. Any preamplification product comprised by the detection mixture can be amplified by PCR. In some embodiments, a detection mixture can comprise (a) a universal primer which hybridizes to the complement of the primer target sequence and (b) the reverse primer which hybridizes to a complement of the target nucleic acid. In some embodiments, the detection mixture can further comprise a detection probe. Amplification can be detected and quantified as described supra. Detecting amplification can utilize any technique well-known to skilled artisans, including various nucleic acid detection methods, such as methods for detecting nucleic acids synthesized during PCR, or indirect methods such as detection of a probe binding to a nucleic acid generated in a PCR. In some embodiments, detecting amplification can comprise detecting using a real-time PCR assay or an end-point PCR assay.

In some embodiments, a first primer target sequence and a second primer target sequence can be identical. In some embodiments, the universal primers can also be identical. In some embodiments, at least one of a 3′ portion of a forward primer that hybridizes to the target nucleic acid and 3′ portion of a reverse primer that hybridizes to a complement of the target nucleic acid can comprise an exon-exon junction-spanning sequence or a complement thereof.

In some embodiments, at least one of the first and the second detection probes can further comprise an electrophoretic mobility modifier. Moreover, in some embodiments, detecting amplification of a preamplification product can comprise detection by use of an electrophoresis assay, such as a capillary electrophoresis assay or a gel electrophoresis assay.

### EXAMPLES

Aspects of the present teachings may be further understood in light of the following examples, which should not be construed as limiting the scope of the present teachings in any way. Use of the past tense in these Examples is not to be understood as an indication that any described event actually occurred or that any reported results were actually achieved.

### Example 1

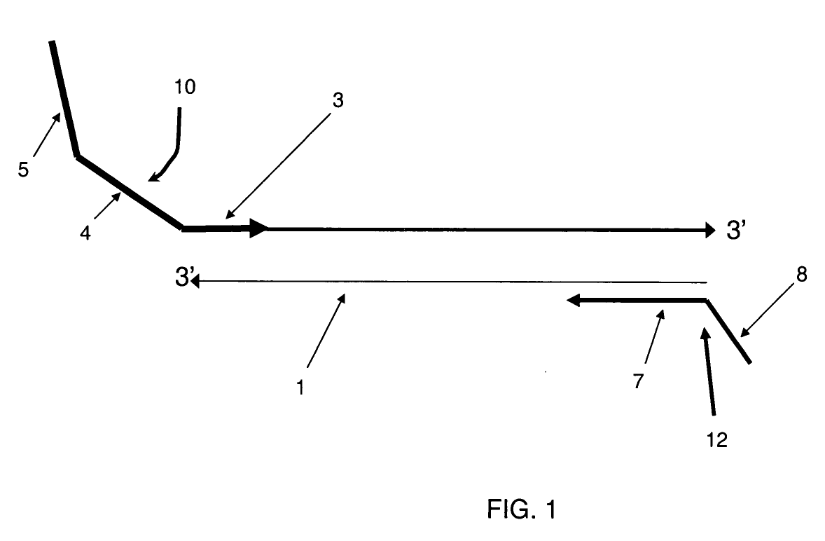

This example illustrates an initial mixture and formation of a preamplification product comprising the sequence of a cDNA, using as an exemplary target cDNA CYP2D6, which is polypeptide 6 of subfamily D of the human polypeptide cytochrome P450 family. In this example, as illustrated in FIG. 1, the forward primer 10 comprises a first primer target sequence 5, a detection probe sequence 4, and a first portion 3 that hybridizes to the target nucleic acid 1. Furthermore, in this example, the reverse primer 12 comprises a second primer sequence 8 and a second portion 7 that hybridizes to the complement of the target sequence.

In this example, the CYP2D6 cDNA 1 has the sequence tatggggctagaagcactggtgccctggccgtgatagtggccatcttcctgctcctggtggacctgatgcaccggcgccaacgctgggctg cacgctacccaccaggcccctgccactgccgggctgggcaacctgctgcatgtggacttccagaacacaccatactgcttcgaccagtt gcggcgccgcttcggggacgtgttcagcctgcagctggcctggacgccggtggtcgtgctcaatgggctggcggccgtgcgcgaggcgct ggtgacccacggcgaggacaccgccgaccgccgcctgtgcccatcacccagatcctgggttcgggccgcgttcccaaggggtgttcct ggcgcgctatgggcccgcgtggcgcgagcagaggcgcttttccgtctccaccttgcgcaacttgggcctgggcaagaagtcgctggagc agtgggtgaccgaggaggccgcctgccttgtgccgccttcgccaaccactccggacgcccctcgcccaacggtctcttggacaaagc cgtgagcaacgtgatcgcctccctcacctgcgggcgccgcttcgagtacgacgaccctcgcttcctcaggctgctggacctagctcaggag ggactgaaggaggagtcgggcttctgcgcgaggtgctgaatgctgtcccgtcctcctgcatatcccagcgctggctggcaaggtcctacg cttccaaaaggcttctgacccagctggatgagctgctaactgagcacaggatgacctgggacccagcccagccccgagacctga ctgaggccttcctggcagagatggagaaggccaaggggaaccctgagagcagcttcaatgatgagaacctgtgcatagtggtggctgac ctgtttcttgccgggatggtgaccacctcgaccacgctggcctggggcctcctgctcatgatcctacatccggatgtgcagcgccgtgtccaa caggagatcgacgacgtgatagggcaggtgcggcgaccagagatgggtgaccaggctcacatgcctacaccactgccgtgattcatg aggtgcagcgcttggggacatcgtcsccctgggtgtgacccatatgacatcccgtgacatcgaagtacagggcttccgcatccctaaggg aacgacactcatcaccaacctgtcatcggtgctgaaggatgaggccgtctgggagaagcccttccgctccacccgaacacttcctggat gcccagggccacttgtgaagccggaggccttcctgcmcttcagcagggcgccgtgcatgcctcggggagcccctggccgcatggag ctcttcctctttctcacctccctgctgcagcacttcagttctctggtgcccactggacagcccggcccagccaccatggtgtcttgmcttctgg tgaccccatccccctatgagcttgtgctgtgcccgctagaatggggtacctagtcccca (SEQ ID NO:1).

The forward primer 10 of this example comprises the sequence tccaccgtggcactataagaaccggctagtccttcaagcgcctggggactaggtaccccattcta (SEQ ID NO:2), wherein the first primer target sequence 5 consists of tccaccgtggcactataagaaccggc (SEQ ID NO:3), the detection probe sequence 4 consists of tagtccttcaagcgcc (SEQ ID NO:4), and the first portion 3, which hybridizes to the cDNA, consists of tggggactaggtaccccattcta (SEQ NO:5). The reverse primer 12 has the sequence agatctgagcgcggctcttatcttatggggctagaagcactggtgcccct (SEQ ID NO:6), wherein the second primer target sequence 8 consists of agatctgagcgcggctcttatct (SEQ ID NO:7), and the second portion 7, which hybridizes to a complement of the target nucleic acid, consists of tatggggctagaagcactggtgcccct (SEQ ID NO:8), which is identical to a sequence of the 5′ end of the cDNA 1.

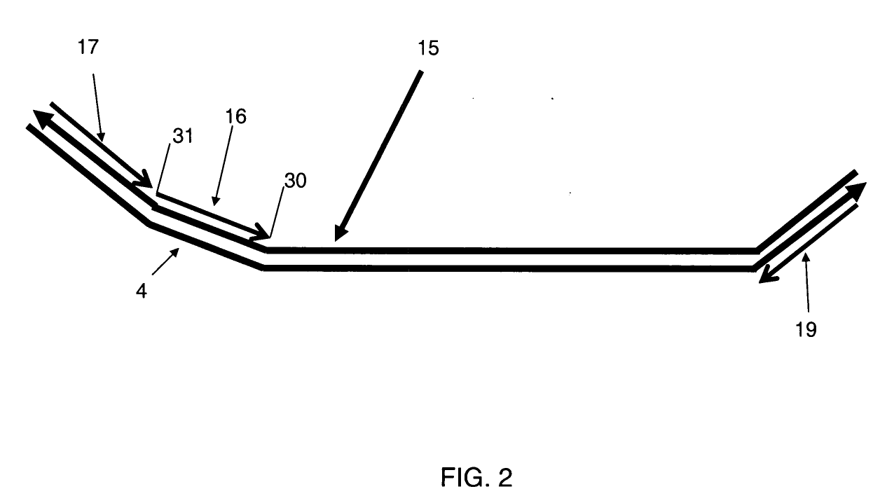

In this example, an initial mixture is prepared on ice by forming a combination comprising: a) cDNA of RNA prepared from patient blood; b) Taq polymerase; c) the forward primer; d) the reverse primer; and, as a PCR reaction mix, (e) TaqMan® Universal Master Mix (Applied Biosystems, Foster City, Calif.) comprising buffer and salts, dNTPs in accordance with well-established PCR protocols, and sterile water. The initial mixture is subjected to 10 cycles of thermal cycling for elongation, wherein each cycle comprises the following temperature conditions: 95° C. for 1 minute; 65° C. for 2 minutes; and 72° C. for 3 minutes. The treatment yields the preamplification product 15, as illustrated in Example 2 and FIG. 2 below.

### Example 2

This example illustrates a detection mixture and nucleic acid detection. In this example, as represented in FIG. 2, one strand of a preamplification product 15, formed in accordance with methods disclosed in Example 1, is illustrated with its 3′ end situated toward the left. A detection mixture in this example comprises: a) the preamplification product 15; b) Taq polymerase; c) a universal forward primer 17 comprising the sequence tccaccgtggcactataagaaccggc (SEQ ID NO:3), which is identical to the first primer target sequence 5 of FIG. 1; d) a universal reverse primer 19, comprising the sequence agatctgagcgcggctcttatct (SEQ ID NO:7), which is identical to the second primer target sequence 8 of FIG. 1; and e) a TaqMan® probe 16 comprising a FAM fluorophore 31 at the probe's 5′ end, an MGB non-fluorescent quencher 30 at the probe's 3′ end, and a detection probe sequence 4 consisting of tagtccttcaagcgcc (SEQ ID NO:4). The detection mixture is subjected to 35 cycles of thermal cycling, wherein each cycle comprises the following temperature conditions: 95° C. for 1 minute; and 72° C. for 4 minutes.

The thermal cycling results in release of FAM fluorophore 31 from the probe as a DNA polymerase hydrolyzes the probe as a result of its 5′-nuclease activity. Accumulation of unquenched FAM fluorophore 31 is measured in real-time by measurement of fluorescence intensity of the mixture using a laser light source for fluorophore excitation and an Applied Biosystems ABI PRISM® 7900HT Sequence Detection System. Threshold cycle (C_{T}) values are determined, and the quantity of the target is calculated based upon comparison with calibration measurements.

### Example 3

This example illustrates initial mixtures and formation of a plurality of preamplification products, wherein the target cDNAs are obtained from different sources and each sample comprises a cDNA of CYP2D6 as an exemplary target cDNA. In this example, each forward primer 10 comprises a first primer target sequence 5 and a first portion 3 which hybridizes to the target nucleic acid, and the reverse primer 12 comprises a second primer sequence 7 and a second portion 8 which hybridizes to the complement of the target sequence. In addition, each forward primer 10 further comprises a unique detection probe sequence 4.

In this example, the CYP2D6 cDNA sequence 1 is identical to the sequence set forth in Example 1. The initial mixtures are each prepared as disclosed in Example 1, wherein each initial mixture includes a cDNA of RNA prepared from blood of one of four human patients. The forward primers 10 each comprise the first primer target sequence 5 disclosed in Example 1 tccaccgtggcactataagaaccggc (SEQ ID NO:3) and the first portion 3 which hybridizes to the cDNA consists of tggggactaggtaccccattcta (SEQ ID NO:5), also as set forth in Example 1. Furthermore, the forward primers 10 each comprise a unique detection probe sequence 4 selected as follows:

| | |
|---|---|
| tagtccttcaagcgac | (SEQ ID NO:9) |
| tagtccttcaagcacc | (SEQ ID NO:10) |
| tagtccttcaagcctc | (SEQ ID NO:11) |
| tagtccttcaagctgc | (SEQ ID NO:12) |

Accordingly, forward primers FP1, FP2, FP3, and FP4 have the following sequences, wherein the detection probe sequences 4 are shown underlined:

FP1:

(SEQ ID NO:13)

tccaccgtggcactataagaaccggctagtccttcaagcacctggggact
aggtaccccattcta
FP2:

(SEQ ID NO:14)

tccaccgtggcactataagaaccggctagtccttcaagcgactggggact
aggtaccccattcta
FP3:

(SEQ ID NO:15)

tccaccgtggcactataagaaccggctagtccttcaagcctctggggact
aggtaccccattcta
FP4:

(SEQ ID NO:16)

tccaccgtggcactataagaaccggctagtccttcaagctgctggggact
aggtaccccattcta
In this example, the reverse primer 12 is the same
for all the initial mixtures, i.e. agatctgagcgcggc
tcttatcttatggggctagaagcactggtgcccct (SEQ ID NO:6)
as set forth in Example 1.

Each initial mixture is prepared and amplified as described in Example 1, yielding four different preamplification products, PP₁ (20), PP₂, PP₃, and PP₄, which differ from one another by no more than two nucleotides within the detection probe sequence.

### Example 4

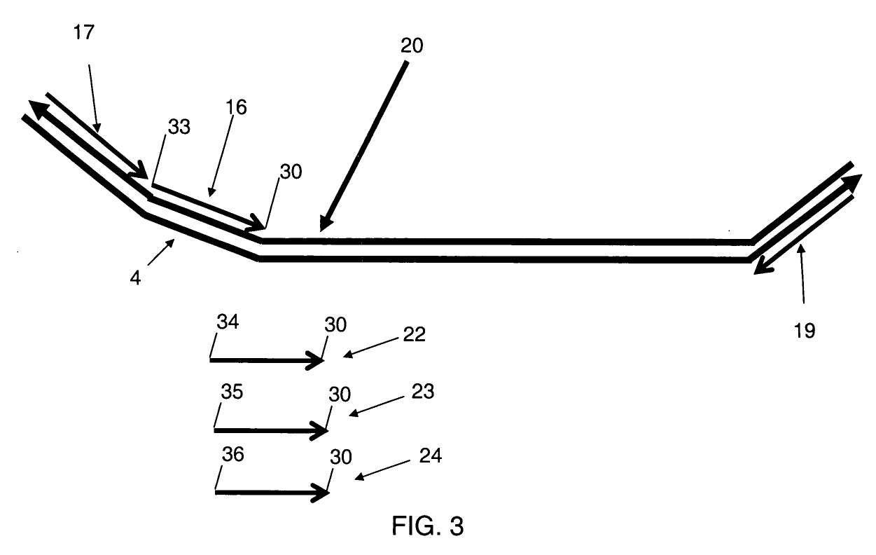

This example illustrates a detection mixture and nucleic acid detection. In this example, as represented in FIG. 3, both strands of preamplification product PP₁ (20), formed in accordance with methods disclosed in Example 3, are illustrated with their 3′ primers. The detection mixture in this example comprises: a) preamplification products PP₁ (20), PP₂, PP₃, and PP₄; b) Taq polymerase; c) a universal forward primer 17 comprising the sequence tccaccgtggcactataagaaccggc (SEQ ID NO:3), which is identical to the first primer target sequence 5 of FIG. 1; d) a universal reverse primer 19, comprising the sequence agatctgagcgcggctcttatct (SEQ ID NO:7), which is identical to the second primer target sequence 7 of FIG. 1; and e) a plurality of TaqMan® probes 16, 22, 23, and 24.

The TaqMan® probes 16, 22, 23, and 24 comprise, respectively, a fluorophore 33, 34, 35 or 36 at their 5′ ends, an MGB non-fluorescent quencher 30 at their 3′ ends, and a nucleotide sequence that is identical to one of the detection probes SEQ ID NO:9 (16), SEQ ID NO:10 (22), SEQ ID NO:11 (23), or SEQ ID NO:12 (24), respectively, listed in Example 3. Fluorophores are FAM 33, VIC 34, HEX 35, and JOE 36. These probes therefore comprise the following structures:

| | |
|---|---|
| 16: FAM-tagtccttcaagcgac-Q | (SEQ ID NO:9) |
| 22: VIC-tagtccttcaagcacc-Q | (SEQ ID NO:10) |
| 23: HEX-tagtccttcaagcctc-Q | (SEQ ID NO:11) |
| 24: JOE-tagtccttcaagctgc-Q | (SEQ ID NO:12) |

The detection mixture is subjected to 35 cycles of thermal cycling, wherein each cycle comprises the following temperature conditions: 96° C. for 1 minute; and 73° C. for 4 minutes.

The thermal cycling results in release of the fluorophores 33, 34, 35, 36 from those TaqMan® probes 16, 22, 23, 24 that hybridized to their targets, as the DNA polymerase hydrolyzes the probes 16, 22, 23, 24 by action of its 5′-nuclease activity. Accumulation of the unquenched fluorophores 33, 34, 35, 36 is measured in real-time by measurement of fluorescence intensity of the mixture for each fluorophore, using a laser light sources for fluorophore excitation, and these values are recorded using an Applied Biosystems ABI PRISM® 7900HT Sequence Detection System. Threshold cycle (C_{T}) values are determined for each fluorophore, and the quantity of the target nucleic acid for each sample is calculated based upon comparison with calibration measurements.

### Example 5

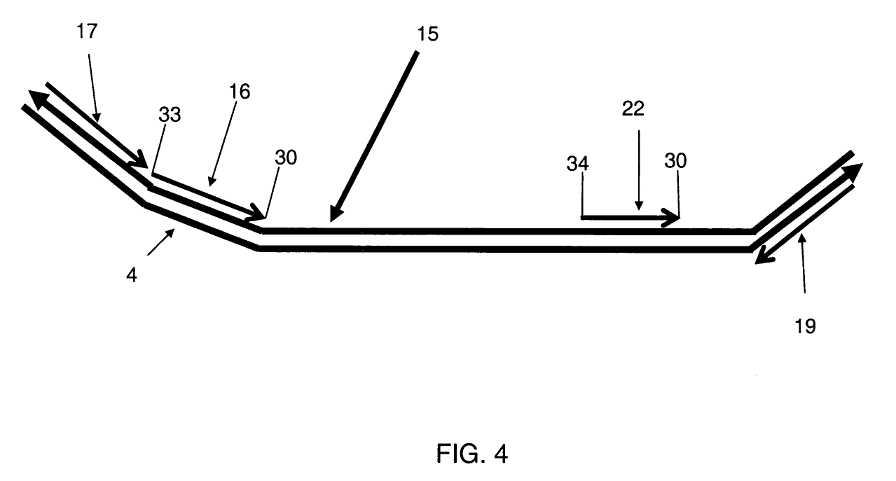

This example illustrates a detection mixture and detection of a nucleic acid. In this example, as represented in FIG. 4, both strands of a preamplification product 15, formed in accordance with methods disclosed in Example 1, are illustrated. A detection mixture in this example comprises: a) the preamplification product 15; b) Taq polymerase; c) a universal forward primer 17 comprising the sequence tccaccgtggcactataagaaccggc (SEQ ID NO:3), which is identical to the first primer target sequence 5; d) a universal reverse primer 19, comprising the sequence agatctgagcgcggctcttatct (SEQ ID NO:7), which is identical to the second primer target sequence 8; and e) a TaqMan® probe 16 and a Molecular Beacon probe 22. The TaqMan® probe 16 comprises a FAM fluorophore 33 at the probe's 5′ end, an MGB non-fluorescent quencher 30 at the probe's 3′ end, and a detection probe sequence 4 consisting of tagtccttcaagcgcc (SEQ ID NO:4). The Molecular Beacon probe 22 comprises a VIC fluorophore 34 at the probe's 5′ end, an MGB non-fluorescent quencher 30 at the probe's 3′ end, and a nucleotide sequence consisting of gcgctggctggcaaggtcctacgc (SEQ ID NO:17), which is a sequence identical to nucleotides 704-727 of the CYP2D6 cDNA. The detection mixture is subjected to 35 cycles of thermal cycling, wherein each cycle comprises the following temperature conditions: 95° C. for 1 minute; and 72° C. for 4 minutes.

The thermal cycling results in release from quenching of FAM fluorophore 33 as the DNA polymerase hydrolyzes the TaqMan® probe 16 as a result of its 5′-nuclease activity. In addition, the thermal cycling results in release from quenching of VIC fluorophore 34 as the Molecular Beacon probe 22 hybridizes to the target nucleic acid as the latter is replicated. Unquenched FAM fluorophore 33 and VIC fluorophore 34 fluorescence levels are measured in real-time by measurement of fluorescence intensities of the fluorophores using a laser light source for fluorophore excitation and an Applied Biosystems ABI PRISM® 7900HT Sequence Detection System. Threshold cycle (C_{T}) values are determined, and the quantity of the target calculated based upon comparison with calibration measurements, in which samples of known quantity are used to construct a standard curve. In addition, fluorescence measurements are normalized for probe and instrumentation differences, such as binding efficiencies and fluorophore quantum yields. A calculated molar ratio of 1:1 for the two unquenched fluorophores 33, 34 indicates that the probes can be used interchangeably for measuring CYP2D6 cDNA sequence 1 in a sample. Furthermore, the TaqMan® probe 16 serves as an internal control for comparison to parallel investigations of other samples in which the CYP2D6 cDNA sequence 1 is measured.

### Example 6

This example illustrates detection of a cDNA 1 in a sample using a primer that spans an exon-exon boundary 25. In this example, examination of sequences contained in a database indicated that CYP2D6 mRNA comprises an exon-exon boundary 25 at nucleotides 181-182, within the sequence cttcgaccagttgcggcgccg (SEQ ID NO:18). Accordingly, a reverse primer 12 for detection of this cDNA 1 comprises, as the second position 7, cggcgccgcaactggtcgaag (SEQ ID NO 19). A complete reverse primer has the sequence agatctgagcgcggctcttatctcggcgccgcaactggtcgaag (SEQ ID NO:20), in which the first 23 nucleotides (agatctgagcgcggctcttatct, SEQ ID NO:7) are identical to the second primer target sequence 8.

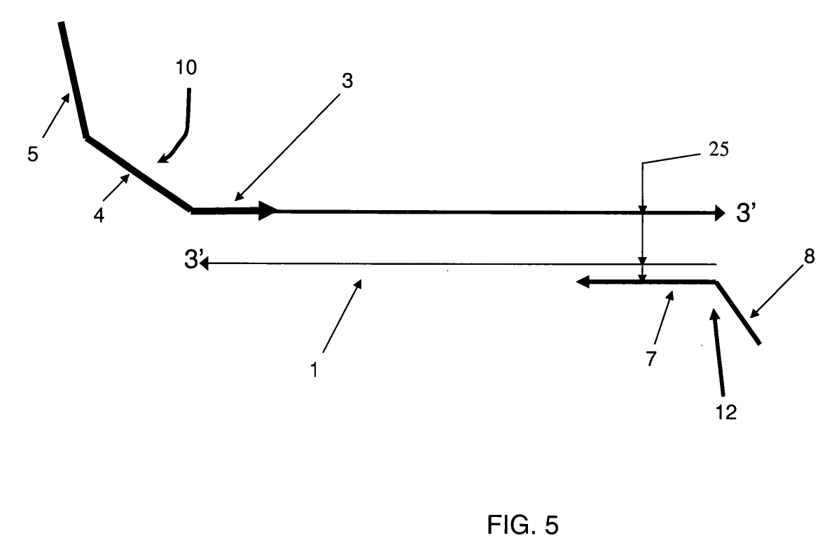

To detect the CYP2D6 cDNA 1, an initial mixture (as shown in FIG. 5) is prepared comprising: the reverse primer 12; the forward primer 10, described in Example 1 and having the sequence, tccaccgtggcactataagaaccggctagtccttcaagcgcctggggactaggtaccccattcta (SEQ ID NO:2); the cDNA 1; and Taq polymerase; as well as TaqMan® Universal Master Mix (Applied Biosystems, Foster City, Calif.) comprising buffer and salts, dNTPs in accordance with well-established PCR protocols, and sterile water. The initial mixture is heated to 60° C. for 2 minutes to promote initial elongation. The initial mixture is then subjected to 10 cycles of thermal cycling, wherein each cycle comprises the following temperature conditions: 95° C. for 1 minute; 65° C. for 2 minutes; and 72° C. for 3 minutes. This treatment yields a preamplification product 20, which is then quantified using the methods described in Example 2, supra. Use of a target-specific probe which comprises an exon-exon junction-spanning sequence 25 reduces potential background signal resulting from genomic DNA that might be comprised by a sample.

### Example 7

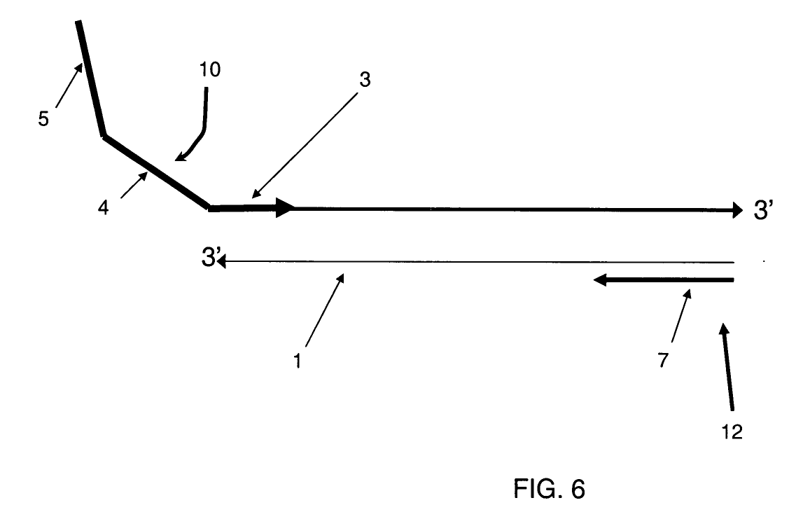

This example illustrates detection of a CYP2D6 cDNA 1 in a sample using: a forward primer 12 comprising a first primer target sequence 5, a probe sequence 4, and a first portion 3 that hybridizes to target nucleic acid sequence 3; and a reverse primer 12 consisting of only a second portion 7 that hybridizes to target nucleic acid sequence. As illustrated in FIG. 6, in this example, an initial mixture is prepared as described in Example 1, except that the reverse primer is a sequence complementary to the first 22 nucleotides from the 5′ region of the CYP2D6, i.e. the complement of tatggggctagaagcactggtg (SEQ ID NO:21), which is caccagtgcttctagccccata (SEQ ID NO:22). Ten cycles of PCR yield a preamplification product comprising a first primer target sequence 5, a detection probe sequence 4, and cDNA sequence 1. This preamplification product is amplified in a detection mixture that comprises a universal primer 17 complementary to the first primer target sequence 5, a TaqMan® detection probe 16, and the same reverse primer 12 used to generate the preamplification product. A real-time PCR assay conducted as described in Example 2 provides quantitative data regarding the presence of CYP2D6 cDNA in the sample.

### Example 8

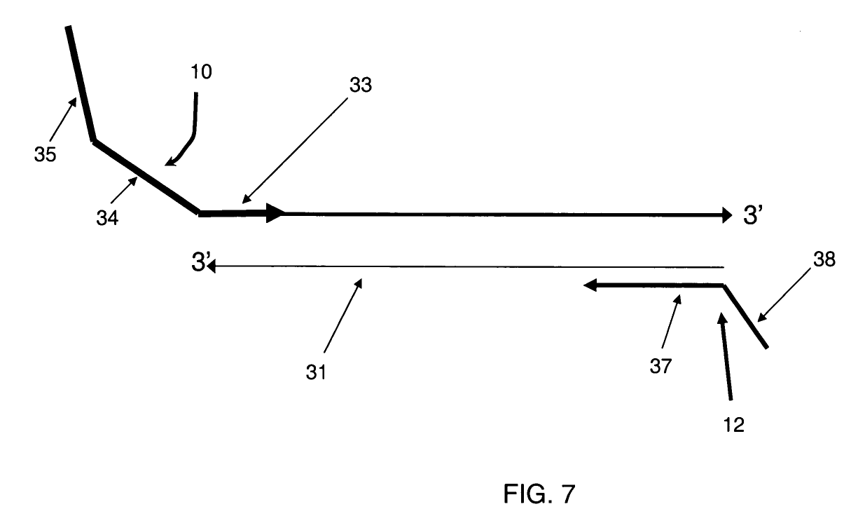

This example illustrates an initial mixture and multiplex preamplification of a plurality of cDNA templates 31 in the mixture. In this example, the multiplex preamplification is a 43-plex reaction, in which the initial mixture includes forward primers 40, listed in Table 4, and reverse primers 42, listed in Table 1. In this example, one of the forward primers 40, noted as FP1, is matched with one of the reverse primers 42, noted as RP1. FP1 and RP1 hybridize to one of the plurality of cDNA templates 31 for preamplification using any of the methods described supra. Another of the forward primers 40, noted as FP2, is matched with another of the reverse primers 42, noted as RP2. FP2 and RP2 hybridize to another of the plurality of cDNA templates 31 for preamplification. Each of the forward primers 40 comprises a first primer target sequence 35 (listed in Table 5), a detection probe sequence 34 (listed in Table 6), and a first portion 33 that hybridizes to the target nucleic acid (listed in Table 7). Furthermore, in this example, the reverse primers 42 comprise a second primer sequence 38 (listed in Table 2) and a second portion 37 that hybridizes to the complement of the target sequence (listed in Table 3), as illustrated in FIG. 7. The first primer target sequence 35 can be a ZipCode™ sequence or can be a universal primer sequence. The second primer sequence 38 can be a ZipCode™ sequence or can be a universal primer sequence.

In this example, the initial mixture is prepared on ice by forming a combination comprising: a) a plurality of cDNAs of RNA prepared from patient blood; b) Taq polymerase; c) the forward primers 40; and d) the reverse primers 42; as well as TaqMan® Universal Master Mix (Applied Biosystems, Foster City, Calif.) comprising buffer and salts, dNTPs in accordance with well-established PCR protocols, and sterile water. The initial mixture is subjected to 10 cycles of thermal cycling for elongation, wherein each cycle comprises the following temperature conditions: 95° C. for 1 minute; 65° C. for 2 minutes; and 72° C. for 3 minutes. The treatment yields a plurality of preamplification products and each of the plurality of preamplification products can be part of a detection mixture and analyzed using methods and examples described supra.

TABLE 1

| | | |
|---|---|---|
| SEQ ID NO | 42 | RP |

| | | |
|---|---|---|
| 23 | AGCATGAACAGCCTAAACACTATAGCGATGCCCAGAAGTTTCT | 1 |
| 24 | AGCATGAAAGTGGGCTTGTGACAAAATACCCTGCCTCTGGTGTA | 2 |
| 25 | AGCATGAAATCCACGTTGTCGTCTCCACGTCAGGCGTTGT | 3 |
| 26 | AGCATGCAAGTCGTAAACCACGGACTCGCAGCCGTGTCT | 4 |
| 27 | AGCATGACGTGCTATAACCATACATGCAGGACACCCAGGTT | 5 |
| 28 | AGCATGCCATCTCCGAACCTATTGGCGTTGTCGAAGTCAGA | 6 |
| 29 | AGCATGCCACATAACAACGAATGTCTGTGTACTCCTTCCCTTCTTG | 7 |
| 30 | AGCATGCAAGGACGTAACGTAGTTTCCTCGTTGTCCTTCTTGAAC | 8 |
| 31 | AGCATGCAATCGCCATTGATGATTTCCTCTGCCAGCAACGT | 9 |
| 32 | AGCATGCACATCAGCAACTTCTGGCGTATTTCTCTTACCAGTGTGT | 10 |
| 33 | AGCATGAACGGTTCAAAGACCCGGTCTTCCTCCGACTCACTA | 11 |
| 34 | AGCATGCAAGAGGCATTCTGACTGGGTCGAATTTGTTGCAA | 12 |
| 35 | AGCATGAGAATCGCCAAGATGGTCCTTTTGATCACTCCCACCTT | 13 |
| 36 | AGCATGCGGGCATAGAAGATCCACCAGGTGGGCCTCTAGGT | 14 |
| 37 | AGCATGACGGAAAACAAGCAAGGCCTTGCTCTTGTTTTCACA | 15 |
| 38 | AGCATGCCGAAAATCAAGCAGACCAGCAGAATGCCAACCA | 16 |
| 39 | AGCATGCCATATTCCAAGCCAACATCGCCCGACGACTTCT | 17 |
| 40 | AGCATGAAGCTGTTCAAGGAATCGCTTGTGTGGGTTAAAAGTCA | 18 |
| 41 | AGCATGCAAGTACCCTTCCTTAGTTCCTGGGAAACAATCAATGAGT | 19 |
| 42 | AGCATGACGAGATTGAAGGGACTCATAGGCGATCCTTTTGATAGC | 20 |
| 43 | AGCATGCAGTCCCTATTCCGAAACTGATAGACGTAATCCCAAAGCA | 21 |
| 44 | AGCATGAAGGCAAAGAAGTCCAAATGGTCGGCTTAGCTTCTTG | 22 |
| 45 | AGCATGCCATGCGTAACAAGTGCCATGATCCTATTTCCTTTTGAG | 23 |
| 46 | AGCATGACATGCCTGACACATTGCATTGTCCAGTTCCCATTC | 24 |
| 47 | AGCATGAAGACGCGATGTTAGGGGAGGTTATATCCTTACCGTACGT | 25 |
| 48 | AGCATGAAAAACCCTTGTGGAGCAGCCTCCCGATTTGG | 26 |
| 49 | AGCATGAAGACCTTCTGTGCGCGGTAGCTCTGTCCATCACCAT | 27 |
| 50 | AGCATGAAAACCAATTGTGCCCTCTTTTGAAACGGTCTTTTAAACG | 28 |
| 51 | AGCATGAGAAACCAGTGTGAGTCAAATCCAACAAAGTCTGGCTTA | 29 |
| 52 | AGCATGCGACCACTAACAGTCTTGTTGTCCCCTCTGACTCTCTCT | 30 |
| 53 | AGCATGCAGTTATCTTGTCCGCAGGGCGGACAGACTTAGCA | 31 |
| 54 | AGCATGAAAGTCGTCTGTAGCCCCACTGGCTGCCAAGAG | 32 |
| 55 | AGCATGCACGGTCAATGTAGATTTTCGGCTTCCTCTCTCTGAA | 33 |
| 56 | AGCATGAGATGACCTTGGTTCGGGCAATCCTACAGCCAAGAG | 34 |
| 57 | AGCATGACAGAACTGACCACGTGTACTTTTTGTGGCTTGGTTTCTCT | 35 |
| 58 | AGCATGCAAAGAATGACCACCCCACTGGGCCCATCTTTAGTATTA | 36 |
| 59 | AGCATGCAAGGAGGCTGGTATCCTGTGATCACATCTTTCTCCTCAT | 37 |
| 60 | AGCATGCGGGAATGAACCATAGCTGGATGGAGGAGAGCTTACAT | 38 |
| 61 | AGCATGAAACCCCTCTGGGTATCATGGATGATGGCCAAGTG | 39 |
| 62 | AGCATGAAAGGGAGGACCCATCCAAAAGCCCCTCTTCCAA | 40 |
| 63 | AGCATGCCAATCTAATGGCTTGCATGAGAACCGCCATTGATG | 41 |
| 64 | AGCATGACAGAGTCAACCGAAAACCGGTGACTTTCTGTTTGGA | 42 |
| 65 | AGCATGAACGAGAATACCGACTGATGTGGTGTTTTCTGGCAAA | 43 |
| 66 | AGCATGACGCTAGAAACCGATAAAGAACAATCCGATGGAAATCTC | 44 |

TABLE 2

| SEQ | | |
|---|---|---|
| ID | | |
| NO | 38 | RP |

| | | |
|---|---|---|
| 67 | AGCATGAACAGCCTAAACACT | 1 |
| 68 | AGCATGAAAGTGGGCTTGTGA | 2 |
| 69 | AGCATGAAATCCACGTTGTCG | 3 |
| 70 | AGCATGCAAGTCGTAAACCAC | 4 |
| 71 | AGCATGACGTGCTATAACCAT | 5 |
| 72 | AGCATGCCATCTCCGAACCTA | 6 |
| 73 | AGCATGCCACATAACAACGAA | 7 |
| 74 | AGCATGCAAGGACGTAACGTA | 8 |
| 75 | AGCATGCAATCGCCATTGATG | 9 |
| 76 | AGCATGCACATCAGCAACTTC | 10 |
| 77 | AGCATGAACGGTTCAAAGACC | 11 |
| 78 | AGCATGCAAGAGGCATTCTGA | 12 |
| 79 | AGCATGAGAATCGCCAAGATG | 13 |
| 80 | AGCATGCGGGCATAGAAGATC | 14 |
| 81 | AGCATGACGGAAAACAAGCAA | 15 |
| 82 | AGCATGCCGAAAATCAAGCAG | 16 |
| 83 | AGCATGCCATATTCCAAGCCA | 17 |
| 84 | AGCATGAAGCTGTTCAAGGAA | 18 |
| 85 | AGCATGCAAGTACCCTTCCTT | 19 |
| 86 | AGCATGACGAGATTGAAGGGA | 20 |
| 87 | AGCATGCAGTCCCTATTCCGA | 21 |
| 88 | AGCATGAAGGCAAAGAAGTCC | 22 |
| 89 | AGCATGCCATGCGTAACAAGT | 23 |
| 90 | AGCATGACATGCCTGACACAT | 24 |
| 91 | AGCATGAAGACGCGATGTTAG | 25 |
| 92 | AGCATGAAAAACCCTTGTGGA | 26 |
| 93 | AGCATGAAGACCTTCTGTGCG | 27 |
| 94 | AGCATGAAAACCAATTGTGCC | 28 |
| 95 | AGCATGAGAAACCAGTGTGAG | 29 |
| 96 | AGCATGCGACCACTAACAGTC | 30 |
| 97 | AGCATGCAGTTATCTTGTCCG | 31 |
| 98 | AGCATGAAAGTCGTCTGTAGC | 32 |
| 99 | AGCATGCACGGTCAATGTAGA | 33 |
| 100 | AGCATGAGATGACCTTGGTTC | 34 |
| 101 | AGCATGACAGAACTGACCACG | 35 |
| 102 | AGCATGCAAAGAATGACCACC | 36 |
| 103 | AGCATGCAAGGAGGCTGGTAT | 37 |
| 104 | AGCATGCGGGAATGAACCATA | 38 |
| 105 | AGCATGAAACCCCTCTGGGTA | 39 |
| 106 | AGCATGAAAGGGAGGACCCAT | 40 |
| 107 | AGCATGCCAATCTAATGGCTT | 41 |
| 108 | AGCATGACAGAGTCAACCGAA | 42 |
| 109 | AGCATGAACGAGAATACCGAC | 43 |
| 110 | AGCATGACGCTAGAAACCGAT | 44 |

TABLE 3

| SEQ | | |
|---|---|---|
| ID | | |
| NO | 37 | RP |

| | | |
|---|---|---|
| 111 | ATAGCGATGCCCAGAAGTTTCT | 1 |
| 112 | CAAAATACCCTGCCTCTGGTGTA | 2 |
| 113 | TCTCCACGTCAGGCGTTGT | 3 |
| 114 | GGACTCGCAGCCGTGTCT | 4 |
| 115 | ACATGCAGGACACCCAGGTT | 5 |
| 116 | TTGGCGTTGTCGAAGTCAGA | 6 |
| 117 | TGTCTGTGTACTCCTTCCCTTCTTG | 7 |
| 118 | GTTTCCTCGTTGTCCTTCTTGAAC | 8 |
| 119 | ATTTCCTCTGCCAGCAACGT | 9 |
| 120 | TGGCGTATTTCTCTTACCAGTGTGT | 10 |
| 121 | CGGTCTTCCTCCGACTCACTA | 11 |
| 122 | CTGGGTCGAATTTGTTGCAA | 12 |
| 123 | GTCCTTTTGATCACTCCCACCTT | 13 |
| 124 | CACCAGGTGGGCCTCTAGGT | 14 |
| 125 | GGCCTTGCTCTTGTTTTCACA | 15 |
| 126 | ACCAGCAGAATGCCAACCA | 16 |
| 127 | ACATCGCCCGACGACTTCT | 17 |
| 128 | TCGCTTGTGTGGGTTAAAAGTCA | 18 |
| 129 | AGTTCCTGGGAAACAATCAATGAGT | 19 |
| 130 | CTCATAGGCGATCCTTTTGATAGC | 20 |
| 131 | AACTGATAGACGTAATCCCAAAGCA | 21 |
| 132 | AAATGGTCGGCTTAGCTTCTTG | 22 |
| 133 | GCCATGATCCTATTTCCTTTTGAG | 23 |
| 134 | TGCATTGTCCAGTTCCCATTC | 24 |
| 135 | GGGAGGTTATATCCTTACCGTACGT | 25 |
| 136 | GCAGCCTCCCGATTTGG | 26 |
| 137 | CGGTAGCTCTGTCCATCACCAT | 27 |
| 138 | CTCTTTTGAAACGGTCTTTTAAACG | 28 |
| 139 | TCAAATCCAACAAAGTCTGGCTTA | 29 |
| 140 | TTGTTGTCCCCTCTGACTCTCTCT | 30 |
| 141 | CAGGGCGGACAGACTTAGCA | 31 |
| 142 | CCCACTGGCTGCCAAGAG | 32 |
| 143 | TTTTCGGCTTCCTCTCTCTGAA | 33 |
| 144 | GGGCAATCCTACAGCCAAGAG | 34 |
| 145 | TGTACTTTTTGTGGCTTGGTTTCTCT | 35 |
| 146 | CCACTGGGCCCATCTTTAGTATTA | 36 |
| 147 | CCTGTGATCACATCTTTCTCCTCAT | 37 |
| 148 | GCTGGATGGAGGAGAGCTTACAT | 38 |
| 149 | TCATGGATGATGGCCAAGTG | 39 |
| 150 | CCAAAAGCCCCTCTTCCAA | 40 |
| 151 | GCATGAGAACCGCCATTGATG | 41 |
| 152 | AACCGGTGACTTTCTGTTTGGA | 42 |
| 153 | TGATGTGGTGTTTTCTGGCAAA | 43 |
| 154 | AAAGAACAATCCGATGGAAATCTC | 44 |

TABLE 4

| SEQ | | |
|---|---|---|
| ID | | |
| NO | 40 | FP |

| | | |
|---|---|---|
| 155 | GGAGCACACCATTAGTTGTGGATCGGCACGCTTCAGACACCGTGTGAATCATTGTCTGACA | 1 |
| 156 | GGAGCAAAACAGGACAACACGATCGGCACGCTTCAGACGTGTGGTCTGGTGCCTGTCTT | 2 |
| 157 | GGAGCACAGAGATGGAACAGAATCGGCACGCTTCAGACCGGCCCTGCGTCATCTC | 3 |
| 158 | GGAGCAACCAAGCACTTGTCAATCGGCACGCTTCAGACAGGAAGGCCCTGGAAATCC | 4 |
| 159 | GGAGCAACCCGTCACTTGTAGATCGGCACGCTTCAGACGGATCTGCGCCGTTTCTGT | 5 |
| 160 | GGAGCACAAACTTCCAACCGTATCGGCACGCTTCAGACTCCTCGTGCGAGAAAGTGAGA | 6 |
| 161 | GGAGCACAAGTTTCGAACCTGATCGGCACGCTTCAGACGCATCATGATGGCCAATTCTG | 7 |
| 162 | GGAGCAAACGAGAACTTGCCCATCGGCACGCTTCAGACGACGATTCTCCACCGAGTATGAG | 8 |
| 163 | GGAGCAACGCACAGTAACTACATCGGCACGCTTCAGACCACGGTGGAGATAGTGACAATGTC | 9 |
| 164 | GGAGCACAACGCTACTTGACAATCGGCACGCTTCAGACTACGGGATTCCATTCATTGAAAC | 10 |
| 165 | GGAGCAAACGCCTGTAAGAAGATCGGCACGCTTCAGACCCTGCGCGCCTACCT | 11 |
| 166 | GGAGCACCACCCTAATTCTTCATCGGCACGCTTCAGACTGCCCAATGCTGACCTTCA | 12 |
| 167 | GGAGCACACTTTGCATTCTCTATCGGCACGCTTCAGACTGCCAAGCTTCTCTGTGAAAGA | 13 |
| 168 | GGAGCAACGTACATCAAGAGGATCGGCACGCTTCAGACTGATGGAAAAGGAGTTGGACTTG | 14 |
| 169 | GGAGCACCCACATCCTTCTAGATCGGCACGCTTCAGACAGAACCAAGACCCAGACATCAAG | 15 |
| 170 | GGAGCACGATCCACATTCTACATCGGCACGCTTCAGACGCCTTGCCCCATCAACTG | 16 |
| 171 | GGAGCACGACCTCGATTCGTAATCGGCACGCTTCAGACGCGCGGCGATCAAGAA | 17 |
| 172 | GGAGCAAGCCTCTCTAAGCTGATCGGCACGCTTCAGACTCGGTGGTACCTGAGATGGA | 18 |
| 173 | GGAGCACAAAAACGGTTCGACATCGGCACGCTTCAGACCACCCTGATGAAAAGATTCAATGAAAACT | 19 |
| 174 | GGAGCACGATGATACTTCCGGATCGGCACGCTTCAGACGCTCACCCTTCCAGACTTCCT | 20 |
| 175 | GGAGCAACAAGGTTTAAGGCTATCGGCACGCTTCAGACACGCCATGCCCATTGG | 21 |
| 176 | GGAGCAACCTACGAGTTCCAAATCGGCACGCTTCAGACAAACCTATAGGCTCAGGAGCTCAAG | 22 |
| 177 | GGAGCACGACTAACATGTTGAATCGGCACGCTTCAGACCTGGAGCGGCCAAGTAGGT | 23 |
| 178 | GGAGCACACACTGGTTGTTCGATCGGCACGCTTCAGACACCCGCTGTATGGAAGGAAAC | 24 |
| 179 | GGAGCAAACGAGTCCTGTTCCATCGGCACGCTTCAGACGGTAGATGGAAAACCGGTGAATC | 25 |
| 180 | GGAGCACCGTTTCTTTGTGTGATCGGCACGCTTCAGACCGAGTCCAAGGCACATATCCA | 26 |
| 181 | GGAGCAAAGCACGTCTGTGTCATCGGCACGCTTCAGACCTGTGACTCCTCCCCAGTATCC | 27 |
| 182 | GGAGCAACAGTTGACACACGGATCGGCACGCTTCAGACCAATTGGAGAAGGACAGCAACAC | 28 |
| 183 | GGAGCACCAGATAAGACACGTATCGGCACGCTTCAGACTGGTCAGGCAGTATAATCCAAAGA | 29 |
| 184 | GGAGCACAGGATACCACAGGAATCGGCACGCTTCAGACCTGGTGGCTGACGACAATCA | 30 |
| 185 | GGAGCAACACTATACACAGGCATCGGCACGCTTCAGACGGTTATCAACTCAGTGGCCTTTG | 31 |
| 186 | GGAGCAAAACGTGAAACATCGATCGGCACGCTTCAGACGCAGTCAACAGCTAAAACCTTCCT | 32 |
| 187 | GGAGCAAGCATCCTCACATCTATCGGCACGCTTCAGACACCTGGCCGAGGACATCAT | 33 |
| 188 | GGAGCACCGTGCATTTGTAAGATCGGCACGCTTCAGACTCCCATCATCTATGCCTTCGTT | 34 |
| 189 | GGAGCACCACCCTTCTGTAACATCGGCACGCTTCAGACTGACAGTGGCAGCTATGAACAAAAT | 35 |
| 190 | GGAGCAAAAAGCGCATGGTTGATCGGCACGCTTCAGACAAGCAGGCTGGCGGAAA | 36 |
| 191 | GGAGCACGGATATTCACCACTATCGGCACGCTTCAGACCTACCTCACTGGCTGGGATGA | 37 |
| 192 | GGAGCAAACAAGAACTGGTCTATCGGCACGCTTCAGACTGAACTTCCCTGGTCGAACAGT | 38 |
| 193 | GGAGCACCATCAAAGACCATTATCGGCACGCTTCAGACTCGGAGCCCTGGAAGCA | 39 |
| 194 | GGAGCAAGAGGCGTCTGGTAAATCGGCACGCTTCAGACAGTTTGCCCGGGAGAGACTT | 40 |
| 195 | GGAGCAAGGAGACTATGGGTCATCGGCACGCTTCAGACCTGGCAACTCTGAAGTCATCCT | 41 |
| 196 | GGAGCAAAAGCTCTCTGGGAGATCGGCACGCTTCAGACCAAGACAATTGTCACCAGGATCAA | 42 |
| 197 | GGAGCACAATTGTGATGGGAAATCGGCACGCTTCAGACAAAAGAATGTCCATGGTGGTGTCT | 43 |
| 198 | GGAGCACACTAACATTGGCTCATCGGCACGCTTCAGACCCACATTCAGCAAGTAGGAAAATTT | 44 |

TABLE 5

| SEQ | | |
|---|---|---|
| ID | | |
| NO | 35 | FP |

| | | |
|---|---|---|
| 199 | GGAGCACACCATTAGTTGTGG | 1 |
| 200 | GGAGCAAAACAGGACAACACG | 2 |
| 201 | GGAGCACAGAGATGGAACAGA | 3 |
| 202 | GGAGCAACCAAGCACTTGTCA | 4 |
| 203 | GGAGCAACCCGTCACTTGTAG | 5 |
| 204 | GGAGCACAAACTTCCAACCGT | 6 |
| 205 | GGAGCACAAGTTTCGAACCTG | 7 |
| 206 | GGAGCAAACGAGAACTTGCCC | 8 |
| 207 | GGAGCAACGCACAGTAACTAC | 9 |
| 208 | GGAGCACAACGCTACTTGACA | 10 |
| 209 | GGAGCAAACGCCTGTAAGAAG | 11 |
| 210 | GGAGCACCACCCTAATTCTTC | 12 |
| 211 | GGAGCACACTTTGCATTCTCT | 13 |
| 212 | GGAGCAACGTACATCAAGAGG | 14 |
| 213 | GGAGCACCCACATCCTTCTAG | 15 |
| 214 | GGAGCACGATCCACATTCTAC | 16 |
| 215 | GGAGCACGACCTCGATTCGTA | 17 |
| 216 | GGAGCAAGCCTCTCTAAGCTG | 18 |
| 217 | GGAGCACAAAAACGGTTCGAC | 19 |
| 218 | GGAGCACGATGATACTTCCGG | 20 |
| 219 | GGAGCAACAAGGTTTAAGGCT | 21 |
| 220 | GGAGCAACCTACGAGTTCCAA | 22 |
| 221 | GGAGCACGACTAACATGTTGA | 23 |
| 222 | GGAGCACACACTGGTTGTTCG | 24 |
| 223 | GGAGCAAACGAGTCCTGTTCC | 25 |
| 224 | GGAGCACCGTTTCTTTGTGTG | 26 |
| 225 | GGAGCAAAGCACGTCTGTGTC | 27 |
| 226 | GGAGCAACAGTTGACACACGG | 28 |
| 227 | GGAGCACCAGATAAGACACGT | 29 |
| 228 | GGAGCACAGGATACCACAGGA | 30 |
| 229 | GGAGCAACACTATACACAGGC | 31 |
| 230 | GGAGCAAAACGTGAAACATCG | 32 |
| 231 | GGAGCAAGCATCCTCACATCT | 33 |
| 232 | GGAGCACCGTGCATTTGTAAG | 34 |
| 233 | GGAGCACCACCCTTCTGTAAC | 35 |
| 234 | GGAGCAAAAAGCGCATGGTTG | 36 |
| 235 | GGAGCACGGATATTCACCACT | 37 |
| 236 | GGAGCAAACAAGAACTGGTCT | 38 |
| 237 | GGAGCACCATCAAAGACCATT | 39 |
| 238 | GGAGCAAGAGGCGTCTGGTAA | 40 |
| 239 | GGAGCAAGGAGACTATGGGTC | 41 |
| 240 | GGAGCAAAAGCTCTCTGGGAG | 42 |
| 241 | GGAGCACAATTGTGATGGGAA | 43 |
| 242 | GGAGCACACTAACATTGGCTC | 44 |

TABLE 6

| SEQ | | |
|---|---|---|
| ID | | |
| NO | 34 | FP |

| | | |
|---|---|---|
| 243 | ATCGGCACGCTTCAGAC | 1 |
| 244 | ATCGGCACGCTTCAGAC | 2 |
| 245 | ATCGGCACGCTTCAGAC | 3 |
| 246 | ATCGGCACGCTTCAGAC | 4 |
| 247 | ATCGGCACGCTTCAGAC | 5 |
| 248 | ATCGGCACGCTTCAGAC | 6 |
| 249 | ATCGGCACGCTTCAGAC | 7 |
| 250 | ATCGGCACGCTTCAGAC | 8 |
| 251 | ATCGGCACGCTTCAGAC | 9 |
| 252 | ATCGGCACGCTTCAGAC | 10 |
| 253 | ATCGGCACGCTTCAGAC | 11 |
| 254 | ATCGGCACGCTTCAGAC | 12 |
| 255 | ATCGGCACGCTTCAGAC | 13 |
| 256 | ATCGGCACGCTTCAGAC | 14 |
| 257 | ATCGGCACGCTTCAGAC | 15 |
| 258 | ATCGGCACGCTTCAGAC | 16 |
| 259 | ATCGGCACGCTTCAGAC | 17 |
| 260 | ATCGGCACGCTTCAGAC | 18 |
| 261 | ATCGGCACGCTTCAGAC | 19 |
| 262 | ATCGGCACGCTTCAGAC | 20 |
| 263 | ATCGGCACGCTTCAGAC | 21 |
| 264 | ATCGGCACGCTTCAGAC | 22 |
| 265 | ATCGGCACGCTTCAGAC | 23 |
| 266 | ATCGGCACGCTTCAGAC | 24 |
| 267 | ATCGGCACGCTTCAGAC | 25 |
| 268 | ATCGGCACGCTTCAGAC | 26 |
| 269 | ATCGGCACGCTTCAGAC | 27 |
| 270 | ATCGGCACGCTTCAGAC | 28 |
| 271 | ATCGGCACGCTTCAGAC | 29 |
| 272 | ATCGGCACGCTTCAGAC | 30 |
| 273 | ATCGGCACGCTTCAGAC | 31 |
| 274 | ATCGGCACGCTTCAGAC | 32 |
| 275 | ATCGGCACGCTTCAGAC | 33 |
| 276 | ATCGGCACGCTTCAGAC | 34 |
| 277 | ATCGGCACGCTTCAGAC | 35 |
| 278 | ATCGGCACGCTTCAGAC | 36 |
| 279 | ATCGGCACGCTTCAGAC | 37 |
| 280 | ATCGGCACGCTTCAGAC | 38 |
| 281 | ATCGGCACGCTTCAGAC | 39 |
| 282 | ATCGGCACGCTTCAGAC | 40 |
| 283 | ATCGGCACGCTTCAGAC | 41 |
| 284 | ATCGGCACGCTTCAGAC | 42 |
| 285 | ATCGGCACGCTTCAGAC | 43 |
| 286 | ATCGGCACGCTTCAGAC | 44 |

TABLE 7

| SEQ | | |
|---|---|---|
| ID | | |
| NO | 33 | FP |

| | | |
|---|---|---|
| 287 | ACCGTGTGAATCATTGTCTGACA | 1 |
| 288 | GTGTGGTCTGGTGCCTGTCTT | 2 |
| 289 | CGGCCCTGCGTCATCTC | 3 |
| 290 | AGGAAGGCCCTGGAAATCC | 4 |
| 291 | GGATCTGCGCCGTTTCTGT | 5 |
| 292 | TCCTCGTGCGAGAAAGTGAGA | 6 |
| 293 | GCATCATGATGGCCAATTCTG | 7 |
| 294 | GACGATTCTCCACCGAGTATGAG | 8 |
| 295 | CACGGTGGAGATAGTGACAATGTC | 9 |
| 296 | TACGGGATTCCATTCATTGAAAC | 10 |
| 297 | CCTGCGCGCCTACCT | 11 |
| 298 | TGCCCAATGCTGACCTTCA | 12 |
| 299 | TGCCAAGCTTCTCTGTGAAAGA | 13 |
| 300 | TGATGGAAAAGGAGTTGGACTTG | 14 |
| 301 | AGAACCAAGACCCAGACATCAAG | 15 |
| 302 | GCCTTGCCCCATCAACTG | 16 |
| 303 | GCGCGGCGATCAAGAA | 17 |
| 304 | TCGGTGGTACCTGAGATGGA | 18 |
| 305 | CACCCTGATGAAAAGATTCAATGAAAACT | 19 |
| 306 | GCTCACCCTTCCAGACTTCCT | 20 |
| 307 | ACGCCATGCCCATTGG | 21 |
| 308 | AAACCTATAGGCTCAGGAGCTCAAG | 22 |
| 309 | CTGGAGCGGCCAAGTAGGT | 23 |
| 310 | ACCCGCTGTATGGAAGGAAAC | 24 |
| 311 | GGTAGATGGAAAACCGGTGAATC | 25 |
| 312 | CGAGTCCAAGGCACATATCCA | 26 |
| 313 | CTGTGACTCCTCCCCAGTATCC | 27 |
| 314 | CAATTGGAGAAGGACAGCAACAC | 28 |
| 315 | TGGTCAGGCAGTATAATCCAAAGA | 29 |
| 316 | CTGGTGGCTGACGACAATCA | 30 |
| 317 | GGTTATCAACTCAGTGGCCTTTG | 31 |
| 318 | GCAGTCAACAGCTAAAACCTTCCT | 32 |
| 319 | ACCTGGCCGAGGACATCAT | 33 |
| 320 | TCCCATCATCTATGCCTTCGTT | 34 |
| 321 | TGACAGTGGCAGCTATGAACAAAAT | 35 |
| 322 | AAGCAGGCTGGCGGAAA | 36 |
| 323 | CTACCTCACTGGCTGGGATGA | 37 |
| 324 | TGAACTTCCCTGGTCGAACAGT | 38 |
| 325 | TCGGAGCCCTGGAAGCA | 39 |
| 326 | AGTTTGCCCGGGAGAGACTT | 40 |
| 327 | CTGGCAACTCTGAAGTCATCCT | 41 |
| 328 | CAAGACAATTGTCACCAGGATCAA | 42 |
| 329 | AAAAGAATGTCCATGGTGGTGTCT | 43 |
| 330 | CCACATTCAGCAAGTAGGAAAATTT | 44 |

### Example 9

This example illustrates detection of a plurality of CYP2D cDNAS 1 using a microcard as a solid support.

In this example, a 96-well microcard is arranged such that each locus comprises a forward primer 10, as set forth in Example 1, except that the reverse primers 12 comprise a biotin moiety at their 5′ ends, and are attached to the loci via streptavidin previously immobilized on the surfaces of the wells of the loci. Forward primer 10 as described in Example 1, Taq polymerase, and other components for PCR are also added to the loci. Samples to be tested are added to individual loci, including positive and negative controls at some loci. The microcard is subjected to 10 cycles of PCR, and preamplification products accumulate, immobilized at the loci where they are formed. The microcard is rinsed, and universal primers 17, 19, a TaqMan® probe 16, and other PCR components are added to all the loci. The microcard is subjected to thermal cycling conditions described in Example 2. The loci are scanned for fluorescence in an end-point assay using an Applied Biosystems ABI PRISM® 7900HT Sequence Detection System. Fluorescence intensity at each locus is indicative of the quantity of CYP2D6 cDNA comprised by each sample.

The examples and other embodiments described herein are exemplary and are not intended to be limiting in describing the full scope of apparatus, systems, compositions, materials, and methods of this teachings. Equivalent changes, modifications, variations in specific embodiments, apparatus, systems, compositions, materials and methods may be made within the scope of the present teachings with substantially similar results. Such changes, modifications or variations are not to be regarded as a departure from the spirit and scope of the teachings.

What is claimed is:

1. A method for detecting a target nucleic acid, the method comprising:
(1) forming an initial mixture by combining components including (a) a sample suspected of comprising the target nucleic acid, (b) a polymerase, (c) a forward primer comprising a 5′ portion comprising a first primer target sequence and a 3′ portion that hybridizes to the target nucleic acid, (d) a reverse primer comprising a 5′ portion comprising a second primer target sequence and a 3′ portion that hybridizes to a complement of the target nucleic acid, and (e) a PCR reaction mix; wherein at least one of the forward primer and the reverse primer further comprises a detection probe sequence; and placing the resulting combination under conditions in which the forward primer elongates, when the target nucleic acid is a cDNA;
(2) forming a preamplification product by subjecting the initial mixture to at least one cycle of a PCR;
(3) forming a detection mixture comprising, in a PCR reaction mix, the preamplification product, a first universal primer that hybridizes to a complement of the first primer target sequence, and a second universal primer that hybridizes to a complement of the second primer target sequence;
(4) amplifying any preamplification product comprised by the detection mixture; and
(5) detecting amplification of any preamplification product, whereby said target nucleic acid is detected, if present in sample (1)(a), by detecting amplification of its preamplification product.

2. The method according to claim 1, wherein the detection mixture further comprises a detection probe comprising a sequence that hybridizes to the detection probe sequence or a complement thereof.

3. The method according to claim 2, wherein the detection probe further comprises a label.

4. The method according to claim 3, wherein the label is selected from the group consisting of fluorophores, biotins, digoxygenin, radioisotopes, and electrophoretic mobility modifiers.

5. The method according to claim 4, wherein the label is a fluorophore.

6. The method according to claim 5, wherein the detection probe further comprises a fluorescence quencher.

7. The method according to claim 5, wherein the fluorophore is selected from the group consisting of FAM, VIC, TET, HEX, JOE, NED, LIZ, TAMRA, ROX, ALEXA, Texas Red, Cy3, Cy5, Cy7, Cy9, and dR6G.

8. The method according to claim 1, wherein at least one of the forward primer and the reverse primer is attached to a solid support.

9. The method according to claim 2, wherein the detection probe is attached to a solid support.

10. The method according to claim 1, wherein at least one of the 3′ portion that hybridizes to the target nucleic acid and 3′ portion that hybridizes to a complement of the target nucleic acid comprises an exon-exon junction-spanning sequence or a complement thereof.

11. The method according to claim 1, wherein the detecting amplification comprises a real-time PCR assay.

12. The method according to claim 1, wherein the forming a preamplification product by subjecting the initial mixture to at least one cycle of a PCR comprises subjecting the initial mixture to from one cycle up to about 10 cycles of a PCR.

13. The method according to claim 1, wherein the detection mixture further comprises a first detection probe comprising a sequence that hybridizes to the detection probe sequence or a complement thereof, and a second detection probe comprising a sequence that hybridizes to a sequence comprised by the target nucleic acid or a complement thereof.

14. The method according to claim 13, wherein the first detection probe comprises a first label and the second detection probe comprises a second label different from the first label, and wherein each label is independently selected from the group consisting of fluorophores, biotins, digoxygenin, radioisotopes, and electrophoretic mobility modifiers.

15. The method according to claim 14, wherein the first label is a fluorophore and the second label is a fluorophore.

16. The method according to claim 15, wherein the first and second fluorophores are each independently selected from the group consisting of FAM, VIC, TET, HEX, JOE, NED, LIZ, TAMRA, ROX, ALEXA, Texas Red, Cy3, Cy5, Cy7, Cy9, and dR6G.

17. A method for detecting a plurality of target nucleic acids, the method comprising:
(1) forming an initial mixture by combining components including (a) a sample suspected of comprising the plurality of target nucleic acids, (b) a polymerase, (c) a plurality of primer sets, each primer set being specific for a different target nucleic acid and each set comprising (i) a forward primer comprising a 5′ portion comprising a first primer target sequence and a 3′ portion that hybridizes to a target nucleic acid of the plurality of nucleic acids, and (ii) a reverse primer comprising a 5′ portion comprising a second primer target sequence and a 3′ portion that hybridizes to a complement of the target nucleic acid, and (d) a PCR reaction mix; wherein at least one of the forward primer and the reverse primer of each primer set further comprises a detection probe sequence unique for the primer set; and placing the resulting combination under conditions in which a forward primer elongates if hybridized to a target nucleic acid;
(2) forming a plurality of preamplification products by subjecting the initial mixture to at least one cycle of a PCR;
(3) forming a detection mixture comprising, in a PCR reaction mix, the plurality of preamplification products, a first universal primer that hybridizes to a complement of the first primer target-sequence, and a second universal primer that hybridizes to a complement of the second primer target sequence;
(4) amplifying any preamplification product comprised by the detection mixture; and
(5) detecting amplification of any preamplification product, whereby said target nucleic acids are detected, if present in sample (1 )(a), by detecting amplification of their preamplification products.

18. The method according to claim 17, wherein the detection mixture further comprises a plurality of detection probes, each detection probe comprising a sequence that hybridizes to a detection probe sequence comprised by a preamplification product or complement thereof.

19. The method according to claim 18, wherein each detection probe further comprises a label.

20. The method according to claim 19, wherein each label is independently selected from the group consisting of fluorophores, biotins, digoxygenin, radioisotopes, and electrophoretic mobility modifiers.

21. The method according to claim 20, wherein each label is a fluorophore.

22. The method according to claim 21, wherein each fluorophore is independently selected from the group consisting of FAM, VIC, TET, HEX, JOE, NED, LIZ, TAMRA, ROX, ALEXA, Texas Red, Cy3, Cy5, Cy7, Cy9, and dR6G.

23. The method according to claim 19, wherein each label is unique to the detection probe.

24. The method according to claim 17, wherein at least one of the forward primer and the reverse primer is attached to a solid support.

25. The method according to claim 17, wherein at least one of the 3′ portion of the forward primer that hybridizes to the target nucleic acid and 3′ portion of the reverse primer that hybridizes to a complement of the target nucleic acid comprises an exon-exon junction-spanning sequence or a complement thereof.

26. The method according to claim 17, wherein the detecting amplification comprises a real-time PCR assay.

27. The method according to claim 17, wherein forming a preamplification product by subjecting the initial mixture to at least one cycle of a PCR comprises subjecting the initial mixture to from one cycle up to about 10 cycles of a PCR.

28. The method according to claim 18, wherein at least one detection probe of the plurality of detection probes is attached to a locus of a solid support comprising a plurality of loci.

28. The method according to claim 27, further comprising contacting the plurality of preamplification products with the plurality of loci.

30. A method of detecting a target nucleic acid in a plurality of samples, the method comprising:
(1) forming a plurality of initial mixtures, by combining, for each initial mixture, components including (a) a sample suspected of comprising the target nucleic acid, (b) a polymerase, (c) a primer set comprising (i) a forward primer comprising a 5′ portion comprising a first primer target sequence and a 3′ portion that hybridizes to the target nucleic acid, and (ii) a reverse primer comprising a 5′ portion comprising a second primer target sequence and a 3′ portion that hybridizes to a complement of the target nucleic acid, and (d) a PCR reaction mix; wherein at least one of the forward primer and the reverse primer further comprises a detection probe sequence unique to each probe set; and placing the resulting combination under conditions in which the forward primer elongates;
(2) forming a plurality of preamplification products by subjecting each initial mixture to at least one cycle of a PCR;
(3) forming a detection mixture comprising, in a PCR reaction mix, the preamplification products, a first universal primer that hybridizes to a complement of the first primer target sequence, and a second universal primer that hybridizes to a complement of the second primer target sequence;
(4) amplifying any preamplification product comprised by the detection mixture; and
(5) detecting amplification of any preamplification product, whereby said target nucleic acid is detected, if present in sample (1)(a), by detecting amplification of its preamplification product.

31. The method according to claim 30, wherein the detection mixture further comprises a plurality of detection probes, each detection probe comprising a sequence that hybridizes to a detection probe sequence or a complement thereof.

32. The method according to claim 31, wherein each detection probe comprises a label.

33. The method according to claim 32, wherein at least one label is a fluorophore.

34. The method according to claim 33, wherein each fluorophore is independently selected from the group consisting of FAM, VIC, TET, HEX, JOE, NED, LIZ, TAMRA, ROX, ALEXA, Texas Red, Cy3, Cy5, Cy7, Cy9, and dR6G.

35. The method according to claim 30, wherein at least one of the 3′ portion of the forward primer that hybridizes to the target nucleic acid and 3′ portion of the reverser primer that hybridizes to a complement of the target nucleic acid comprises an exon-exon junction-spanning sequence or a complement thereof.

36. The method according to claim 30, wherein forming a plurality of preamplification products comprises subjecting the plurality of initial mixture to from one cycle up to about 10 cycles of a PCR.

37. A method for detecting a target nucleic acid, the method comprising:
(1) forming an initial mixture by combining components including (a) a sample suspected of comprising the target nucleic acid, (b) a polymerase, (c) a forward primer comprising a 5′ portion comprising a primer target sequence, a detection probe sequence portion, and a 3′ portion that hybridizes to the target nucleic acid, (d) a reverse primer that hybridizes to a complement of the target nucleic acid; and (e) a PCR reaction mix; and placing the resulting combination under conditions in which the forward primer elongates;
(2) forming a preamplification product by subjecting the initial mixture to at least one cycle of a PCR under annealing conditions in which the forward primer and reverse primer each hybridize to the target nucleic acid or the complement thereof;
(3) forming a detection mixture comprising, in a PCR reaction mix, the preamplification product, a universal primer that hybridizes to a complement of the primer target sequence, and the reverse primer;
(4) amplifying any preamplification product comprised by the detection mixture; and
(5) detecting amplification of any preamplification product, whereby said target nucleic acid is detected, if present in sample (1)(a), by detecting amplification of its preamplification product.

38. The method according to claim 37, wherein the detection mixture further comprises a detection probe comprising a sequence which hybridizes to the detection probe sequence or a complement thereof.

39. The method according to claim 35, wherein at least one of the forward primer and the reverse primer is attached to a solid support.

40. The method according to claim 38, wherein the detection probe is attached to a solid support.

41. The method according to claim 37, wherein the detecting amplification comprises a real-time PCR assay.

42. The method according to claim 37, wherein the forming a preamplification product by subjecting the initial mixture to at least one cycle of a PCR comprises subjecting the initial mixture to from one cycle up to about 10 cycles of a PCR.


SEQUENCE LISTING (330 sequences)

SEQ ID NO: 1 - DNA, 1515 bp, Human
tatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtctccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgtgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgaccccatccccctatgagctttgtgctgtgccccgctagaatggggtacctagtcccca

SEQ ID NO: 2 - DNA, 65 bp, Artificial
  Synthetically generated primer
tccaccgtggcactataagaaccggctagtccttcaagcgcctggggactaggtaccccattcta

SEQ ID NO: 3 - DNA, 26 bp, Human
tccaccgtggcactataagaaccggc

SEQ ID NO: 4 - DNA, 16 bp, Artificial
  Synthetically generated detection probe
tagtccttcaagcgcc

SEQ ID NO: 5 - DNA, 23 bp, Human
tggggactaggtaccccattcta

SEQ ID NO: 6 - DNA, 50 bp, Artificial
  Synthetically generated primer
agatctgagcgcggctcttatcttatggggctagaagcactggtgcccct

SEQ ID NO: 7 - DNA, 23 bp, Human
agatctgagcgcggctcttatct

SEQ ID NO: 8 - DNA, 27 bp, Human
tatggggctagaagcactggtgcccct

SEQ ID NO: 9 - DNA, 16 bp, Artificial
  Synthetically generated probe
tagtccttcaagcgac

SEQ ID NO: 10 - DNA, 16 bp, Artificial
  Synthetically generated probe
tagtccttcaagcacc

SEQ ID NO: 11 - DNA, 16 bp, Artificial
  Synthetically generated probe
tagtccttcaagcctc

SEQ ID NO: 12 - DNA, 16 bp, Artificial
  Synthetically generated probe
tagtccttcaagctgc

SEQ ID NO: 13 - DNA, 65 bp, Artificial
  Synthetically generated primer
tccaccgtggcactataagaaccggctagtccttcaagcacctggggactaggtaccccattcta

SEQ ID NO: 14 - DNA, 65 bp, Artificial
  Synthetically generated primer
tccaccgtggcactataagaaccggctagtccttcaagcgactggggactaggtaccccattcta

SEQ ID NO: 15 - DNA, 65 bp, Artificial
  Synthetically generated primer
tccaccgtggcactataagaaccggctagtccttcaagcctctggggactaggtaccccattcta

SEQ ID NO: 16 - DNA, 65 bp, Artificial
  Synthetically generated primer
tccaccgtggcactataagaaccggctagtccttcaagctgctggggactaggtaccccattcta

SEQ ID NO: 17 - DNA, 24 bp, Artificial
  Synthetically generated probe
gcgctggctggcaaggtcctacgc

SEQ ID NO: 18 - DNA, 21 bp, Human
cttcgaccagttgcggcgccg

SEQ ID NO: 19 - DNA, 21 bp, Artificial
  Synthetically generated primer
cggcgccgcaactggtcgaag

SEQ ID NO: 20 - DNA, 44 bp, Artificial
  Synthetically generated primer
agatctgagcgcggctcttatctcggcgccgcaactggtcgaag

SEQ ID NO: 21 - DNA, 22 bp, Human
tatggggctagaagcactggtg

SEQ ID NO: 22 - DNA, 22 bp, Artificial
  Synthetically generated primer
caccagtgcttctagccccata

SEQ ID NO: 23 - DNA, 43 bp, Artificial
  synthetically derived reverse primer
agcatgaacagcctaaacactatagcgatgcccagaagtttct

SEQ ID NO: 24 - DNA, 44 bp, Artificial
  synthetically derived reverse primer
agcatgaaagtgggcttgtgacaaaataccctgcctctggtgta

SEQ ID NO: 25 - DNA, 40 bp, Artificial
  synthetically derived reverse primer
agcatgaaatccacgttgtcgtctccacgtcaggcgttgt

SEQ ID NO: 26 - DNA, 39 bp, Artificial
  synthetically derived reverse primer
agcatgcaagtcgtaaaccacggactcgcagccgtgtct

SEQ ID NO: 27 - DNA, 41 bp, Artificial
  synthetically derived reverse primer
agcatgacgtgctataaccatacatgcaggacacccaggtt

SEQ ID NO: 28 - DNA, 41 bp, Artificial
  synthetically derived reverse primer
agcatgccatctccgaacctattggcgttgtcgaagtcaga

SEQ ID NO: 29 - DNA, 46 bp, Artificial
  synthetically derived reverse primer
agcatgccacataacaacgaatgtctgtgtactccttcccttcttg

SEQ ID NO: 30 - DNA, 45 bp, Artificial
  synthetically derived reverse primer
agcatgcaaggacgtaacgtagtttcctcgttgtccttcttgaac

SEQ ID NO: 31 - DNA, 41 bp, Artificial
  synthetically derived reverse primer
agcatgcaatcgccattgatgatttcctctgccagcaacgt

SEQ ID NO: 32 - DNA, 46 bp, Artificial
  synthetically derived reverse primer
agcatgcacatcagcaacttctggcgtatttctcttaccagtgtgt

SEQ ID NO: 33 - DNA, 42 bp, Artificial
  synthetically derived reverse primer
agcatgaacggttcaaagacccggtcttcctccgactcacta

SEQ ID NO: 34 - DNA, 41 bp, Artificial
  synthetically derived reverese primer
agcatgcaagaggcattctgactgggtcgaatttgttgcaa

SEQ ID NO: 35 - DNA, 44 bp, Artificial
  synthetically derived reverse primer
agcatgagaatcgccaagatggtccttttgatcactcccacctt

SEQ ID NO: 36 - DNA, 41 bp, Artificial
  synthetically derived reverse primer
agcatgcgggcatagaagatccaccaggtgggcctctaggt

SEQ ID NO: 37 - DNA, 42 bp, Artificial
  synthetically derived reverse primer
agcatgacggaaaacaagcaaggccttgctcttgttttcaca

SEQ ID NO: 38 - DNA, 40 bp, Artificial
  synthetically derived reverse primer
agcatgccgaaaatcaagcagaccagcagaatgccaacca

SEQ ID NO: 39 - DNA, 40 bp, Artificial
  synthetically derived reverse primer
agcatgccatattccaagccaacatcgcccgacgacttct

SEQ ID NO: 40 - DNA, 44 bp, Artificial
  synthetically derived reverse primer
agcatgaagctgttcaaggaatcgcttgtgtgggttaaaagtca

SEQ ID NO: 41 - DNA, 46 bp, Artificial
  synthetically derived reverse primer
agcatgcaagtacccttccttagttcctgggaaacaatcaatgagt

SEQ ID NO: 42 - DNA, 45 bp, Artificial
  synthetically derived reverse primer
agcatgacgagattgaagggactcataggcgatccttttgatagc

SEQ ID NO: 43 - DNA, 46 bp, Artificial
  synthetically derived reverse primer
agcatgcagtccctattccgaaactgatagacgtaatcccaaagca

SEQ ID NO: 44 - DNA, 43 bp, Artificial
  synthetically derived reverse primer
agcatgaaggcaaagaagtccaaatggtcggcttagcttcttg

SEQ ID NO: 45 - DNA, 45 bp, Artificial
  synthetically derived reverse primer
agcatgccatgcgtaacaagtgccatgatcctatttccttttgag

SEQ ID NO: 46 - DNA, 42 bp, Artificial
  synthetically derived reverse primer
agcatgacatgcctgacacattgcattgtccagttcccattc

SEQ ID NO: 47 - DNA, 46 bp, Artificial
  synthetically derived reverse primer
agcatgaagacgcgatgttaggggaggttatatccttaccgtacgt

SEQ ID NO: 48 - DNA, 38 bp, Artificial
  synthetically derived reverse primer
agcatgaaaaacccttgtggagcagcctcccgatttgg

SEQ ID NO: 49 - DNA, 43 bp, Artificial
  synthetically derived reverse primer
agcatgaagaccttctgtgcgcggtagctctgtccatcaccat

SEQ ID NO: 50 - DNA, 46 bp, Artificial
  synthetically derived reverse primer
agcatgaaaaccaattgtgccctcttttgaaacggtcttttaaacg

SEQ ID NO: 51 - DNA, 45 bp, Artificial
  synthetically derived reverse primer
agcatgagaaaccagtgtgagtcaaatccaacaaagtctggctta

SEQ ID NO: 52 - DNA, 45 bp, Artificial
  synthetically derived reverse primer
agcatgcgaccactaacagtcttgttgtcccctctgactctctct

SEQ ID NO: 53 - DNA, 41 bp, Artificial
  synthetically derived reverse primer
agcatgcagttatcttgtccgcagggcggacagacttagca

SEQ ID NO: 54 - DNA, 39 bp, Artificial
  synthetically derived reverse primer
agcatgaaagtcgtctgtagccccactggctgccaagag

SEQ ID NO: 55 - DNA, 43 bp, Artificial
  synthetically derived reverse primer
agcatgcacggtcaatgtagattttcggcttcctctctctgaa

SEQ ID NO: 56 - DNA, 42 bp, Artificial
  synthetically derived reverse primer
agcatgagatgaccttggttcgggcaatcctacagccaagag

SEQ ID NO: 57 - DNA, 47 bp, Artificial
  synthetically derived reverse primer
agcatgacagaactgaccacgtgtactttttgtggcttggtttctct

SEQ ID NO: 58 - DNA, 45 bp, Artificial
  synthetically derived reverse primer
agcatgcaaagaatgaccaccccactgggcccatctttagtatta

SEQ ID NO: 59 - DNA, 46 bp, Artificial
  synthetically derived reverse primer
agcatgcaaggaggctggtatcctgtgatcacatctttctcctcat

SEQ ID NO: 60 - DNA, 44 bp, Artificial
  synthetically derived reverse primer
agcatgcgggaatgaaccatagctggatggaggagagcttacat

SEQ ID NO: 61 - DNA, 41 bp, Artificial
  synthetically derived reverse primer
agcatgaaacccctctgggtatcatggatgatggccaagtg

SEQ ID NO: 62 - DNA, 40 bp, Artificial
  synthetically derived reverse primer
agcatgaaagggaggacccatccaaaagcccctcttccaa

SEQ ID NO: 63 - DNA, 42 bp, Artificial
  synthetically derived reverse primer
agcatgccaatctaatggcttgcatgagaaccgccattgatg

SEQ ID NO: 64 - DNA, 43 bp, Artificial
  synthetically derived reverse primer
agcatgacagagtcaaccgaaaaccggtgactttctgtttgga

SEQ ID NO: 65 - DNA, 43 bp, Artificial
  synthetically derived reverse primer
agcatgaacgagaataccgactgatgtggtgttttctggcaaa

SEQ ID NO: 66 - DNA, 45 bp, Artificial
  synthetically derived reverse primer
agcatgacgctagaaaccgataaagaacaatccgatggaaatctc

SEQ ID NO: 67 - DNA, 21 bp, Artificial
  synthetically derived universal reverse primer
agcatgaacagcctaaacact

SEQ ID NO: 68 - DNA, 21 bp, Artificial
  synthetically derived universal reverse primer
agcatgaaagtgggcttgtga

SEQ ID NO: 69 - DNA, 21 bp, Artificial
  synthetically derived universal reverse primer
agcatgaaatccacgttgtcg

SEQ ID NO: 70 - DNA, 21 bp, Artificial
  synthetically derived universal reverse primer
agcatgcaagtcgtaaaccac

SEQ ID NO: 71 - DNA, 21 bp, Artificial
  synthetically derived universal reverse primer
agcatgacgtgctataaccat

SEQ ID NO: 72 - DNA, 21 bp, Artificial
  synthetically derived universal reverse primer
agcatgccatctccgaaccta

SEQ ID NO: 73 - DNA, 21 bp, Artificial
  synthetically derived universal reverse primer
agcatgccacataacaacgaa

SEQ ID NO: 74 - DNA, 21 bp, Artificial
  synthetically derived universal reverse primer
agcatgcaaggacgtaacgta

SEQ ID NO: 75 - DNA, 21 bp, Artificial
  synthetically derived universal reverse primer
agcatgcaatcgccattgatg

SEQ ID NO: 76 - DNA, 21 bp, Artificial
  synthetically derived universal reverse primer
agcatgcacatcagcaacttc

SEQ ID NO: 77 - DNA, 21 bp, Artificial
  synthetically derived universal reverse primer
agcatgaacggttcaaagacc

SEQ ID NO: 78 - DNA, 21 bp, Artificial
  synthetically derived universal reverse primer
agcatgcaagaggcattctga

SEQ ID NO: 79 - DNA, 21 bp, Artificial
  synthetically derived universal reverse primer
agcatgagaatcgccaagatg

SEQ ID NO: 80 - DNA, 21 bp, Artificial
  synthetically derived universal reverse primer
agcatgcgggcatagaagatc

SEQ ID NO: 81 - DNA, 21 bp, Artificial
  synthetically derived universal reverse primer
agcatgacggaaaacaagcaa

SEQ ID NO: 82 - DNA, 21 bp, Artificial
  synthetically derived universal reverse primer
agcatgccgaaaatcaagcag

SEQ ID NO: 83 - DNA, 21 bp, Artificial
  synthetically derived universal reverse primer
agcatgccatattccaagcca

SEQ ID NO: 84 - DNA, 21 bp, Artificial
  synthetically derived universal reverse primer
agcatgaagctgttcaaggaa

SEQ ID NO: 85 - DNA, 21 bp, Artificial
  synthetically derived universal reverse primer
agcatgcaagtacccttcctt

SEQ ID NO: 86 - DNA, 21 bp, Artificial
  synthetically derived universal reverse primer
agcatgacgagattgaaggga

SEQ ID NO: 87 - DNA, 21 bp, Artificial
  synthetically derived universal reverse primer
agcatgcagtccctattccga

SEQ ID NO: 88 - DNA, 21 bp, Artificial
  synthetically derived universal reverse primer
agcatgaaggcaaagaagtcc

SEQ ID NO: 89 - DNA, 21 bp, Artificial
  synthetically derived universal reverse primer
agcatgccatgcgtaacaagt

SEQ ID NO: 90 - DNA, 21 bp, Artificial
  synthetically derived universal reverse primer
agcatgacatgcctgacacat

SEQ ID NO: 91 - DNA, 21 bp, Artificial
  synthetically derived universal reverse primer
agcatgaagacgcgatgttag

SEQ ID NO: 92 - DNA, 21 bp, Artificial
  synthetically derived universal reverse primer
agcatgaaaaacccttgtgga

SEQ ID NO: 93 - DNA, 21 bp, Artificial
  synthetically derived universal reverse primer
agcatgaagaccttctgtgcg

SEQ ID NO: 94 - DNA, 21 bp, Artificial
  synthetically derived universal reverse primer
agcatgaaaaccaattgtgcc

SEQ ID NO: 95 - DNA, 21 bp, Artificial
  synthetically derived universal reverse primer
agcatgagaaaccagtgtgag

SEQ ID NO: 96 - DNA, 21 bp, Artificial
  synthetically derived universal reverse primer
agcatgcgaccactaacagtc

SEQ ID NO: 97 - DNA, 21 bp, Artificial
  synthetically derived universal reverse primer
agcatgcagttatcttgtccg

SEQ ID NO: 98 - DNA, 21 bp, Artificial
  synthetically derived universal reverse primer
agcatgaaagtcgtctgtagc

SEQ ID NO: 99 - DNA, 21 bp, Artificial
  synthetically derived universal reverse primer
agcatgcacggtcaatgtaga

SEQ ID NO: 100 - DNA, 21 bp, Artificial
  synthetically derived universal reverse primer
agcatgagatgaccttggttc

SEQ ID NO: 101 - DNA, 21 bp, Artificial
  synthetically derived universal reverse primer
agcatgacagaactgaccacg

SEQ ID NO: 102 - DNA, 21 bp, Artificial
  synthetically derived universal reverse primer
agcatgcaaagaatgaccacc

SEQ ID NO: 103 - DNA, 21 bp, Artificial
  synthetically derived universal reverse primer
agcatgcaaggaggctggtat

SEQ ID NO: 104 - DNA, 21 bp, Artificial
  synthetically derived universal reverse primer
agcatgcgggaatgaaccata

SEQ ID NO: 105 - DNA, 21 bp, Artificial
  synthetically derived universal reverse primer
agcatgaaacccctctgggta

SEQ ID NO: 106 - DNA, 21 bp, Artificial
  synthetically derived universal reverse primer
agcatgaaagggaggacccat

SEQ ID NO: 107 - DNA, 21 bp, Artificial
  synthetically derived universal reverse primer
agcatgccaatctaatggctt

SEQ ID NO: 108 - DNA, 21 bp, Artificial
  synthetically derived universal reverse primer
agcatgacagagtcaaccgaa

SEQ ID NO: 109 - DNA, 21 bp, Artificial
  synthetically derived universal reverse primer
agcatgaacgagaataccgac

SEQ ID NO: 110 - DNA, 21 bp, Artificial
  synthetically derived universal reverse primer
agcatgacgctagaaaccgat

SEQ ID NO: 111 - DNA, 22 bp, Artificial
  synthetically derived complement of the target sequence
atagcgatgcccagaagtttct

SEQ ID NO: 112 - DNA, 23 bp, Artificial
  synthetically derived complement of the target sequence
caaaataccctgcctctggtgta

SEQ ID NO: 113 - DNA, 19 bp, Artificial
  synthetically derived complement of the target sequence
tctccacgtcaggcgttgt

SEQ ID NO: 114 - DNA, 18 bp, Artificial
  synthetically derived complement of the target sequence
ggactcgcagccgtgtct

SEQ ID NO: 115 - DNA, 20 bp, Artificial
  synthetically derived complement of the target sequence
acatgcaggacacccaggtt

SEQ ID NO: 116 - DNA, 20 bp, Artificial
  synthetically derived complement of the target sequence
ttggcgttgtcgaagtcaga

SEQ ID NO: 117 - DNA, 25 bp, Artificial
  synthetically derived complement of the target sequence
tgtctgtgtactccttcccttcttg

SEQ ID NO: 118 - DNA, 24 bp, Artificial
  synthetically derived complement of the target sequence
gtttcctcgttgtccttcttgaac

SEQ ID NO: 119 - DNA, 20 bp, Artificial
  synthetically derived complement of the target sequence
atttcctctgccagcaacgt

SEQ ID NO: 120 - DNA, 25 bp, Artificial
  synthetically derived complement of the target sequence
tggcgtatttctcttaccagtgtgt

SEQ ID NO: 121 - DNA, 21 bp, Artificial
  synthetically derived complement of the target sequence
cggtcttcctccgactcacta

SEQ ID NO: 122 - DNA, 20 bp, Artificial
  synthetically derived complement of the target sequence
ctgggtcgaatttgttgcaa

SEQ ID NO: 123 - DNA, 23 bp, Artificial
  synthetically derived complement of the target sequence
gtccttttgatcactcccacctt

SEQ ID NO: 124 - DNA, 20 bp, Artificial
  synthetically derived complement of the target sequence
caccaggtgggcctctaggt

SEQ ID NO: 125 - DNA, 21 bp, Artificial
  synthetically derived complement of the target sequence
ggccttgctcttgttttcaca

SEQ ID NO: 126 - DNA, 19 bp, Artificial
  synthetically derived complement of the target sequence
accagcagaatgccaacca

SEQ ID NO: 127 - DNA, 19 bp, Artificial
  synthetically derived complement of the target sequence
acatcgcccgacgacttct

SEQ ID NO: 128 - DNA, 23 bp, Artificial
  synthetically derived complement of the target sequence
tcgcttgtgtgggttaaaagtca

SEQ ID NO: 129 - DNA, 25 bp, Artificial
  synthetically derived complement of the target sequence
agttcctgggaaacaatcaatgagt

SEQ ID NO: 130 - DNA, 24 bp, Artificial
  synthetically derived complement of the target sequence
ctcataggcgatccttttgatagc

SEQ ID NO: 131 - DNA, 25 bp, Artificial
  synthetically derived complement of the target sequence
aactgatagacgtaatcccaaagca

SEQ ID NO: 132 - DNA, 22 bp, Artificial
  synthetically derived complement of the target sequence
aaatggtcggcttagcttcttg

SEQ ID NO: 133 - DNA, 24 bp, Artificial
  synthetically derived complement of the target sequence
gccatgatcctatttccttttgag

SEQ ID NO: 134 - DNA, 21 bp, Artificial
  synthetically derived complement of the target sequence
tgcattgtccagttcccattc

SEQ ID NO: 135 - DNA, 25 bp, Artificial
  synthetically derived complement of the target sequence
gggaggttatatccttaccgtacgt

SEQ ID NO: 136 - DNA, 17 bp, Artificial
  synthetically derived complement of the target sequence
gcagcctcccgatttgg

SEQ ID NO: 137 - DNA, 22 bp, Artificial
  synthetically derived complement of the target sequence
cggtagctctgtccatcaccat

SEQ ID NO: 138 - DNA, 25 bp, Artificial
  synthetically derived complement of the target sequence
ctcttttgaaacggtcttttaaacg

SEQ ID NO: 139 - DNA, 24 bp, Artificial
  synthetically derived complement of the target sequence
tcaaatccaacaaagtctggctta

SEQ ID NO: 140 - DNA, 24 bp, Artificial
  synthetically derived complement of the target sequence
ttgttgtcccctctgactctctct

SEQ ID NO: 141 - DNA, 20 bp, Artificial
  synthetically derived complement of the target sequence
cagggcggacagacttagca

SEQ ID NO: 142 - DNA, 18 bp, Artificial
  synthetically derived complement of the target sequence
cccactggctgccaagag

SEQ ID NO: 143 - DNA, 22 bp, Artificial
  synthetically derived complement of the target sequence
ttttcggcttcctctctctgaa

SEQ ID NO: 144 - DNA, 21 bp, Artificial
  synthetically derived complement of the target sequence
gggcaatcctacagccaagag

SEQ ID NO: 145 - DNA, 26 bp, Artificial
  synthetically derived complement of the target sequence
tgtactttttgtggcttggtttctct

SEQ ID NO: 146 - DNA, 24 bp, Artificial
  synthetically derived complement of the target sequence
ccactgggcccatctttagtatta

SEQ ID NO: 147 - DNA, 25 bp, Artificial
  synthetically derived complement of the target sequence
cctgtgatcacatctttctcctcat

SEQ ID NO: 148 - DNA, 23 bp, Artificial
  synthetically derived complement of the target sequence
gctggatggaggagagcttacat

SEQ ID NO: 149 - DNA, 20 bp, Artificial
  synthetically derived complement of the target sequence
tcatggatgatggccaagtg

SEQ ID NO: 150 - DNA, 19 bp, Artificial
  synthetically derived complement of the target sequence
ccaaaagcccctcttccaa

SEQ ID NO: 151 - DNA, 21 bp, Artificial
  synthetically derived complement of the target sequence
gcatgagaaccgccattgatg

SEQ ID NO: 152 - DNA, 22 bp, Artificial
  synthetically derived complement of the target sequence
aaccggtgactttctgtttgga

SEQ ID NO: 153 - DNA, 22 bp, Artificial
  synthetically derived complement of the target sequence
tgatgtggtgttttctggcaaa

SEQ ID NO: 154 - DNA, 24 bp, Artificial
  synthetically derived complement of the target sequence
aaagaacaatccgatggaaatctc

SEQ ID NO: 155 - DNA, 61 bp, Artificial
  synthetically derived forward primer
ggagcacaccattagttgtggatcggcacgcttcagacaccgtgtgaatcattgtctgaca

SEQ ID NO: 156 - DNA, 59 bp, Artificial
  synthetically derived forward primer
ggagcaaaacaggacaacacgatcggcacgcttcagacgtgtggtctggtgcctgtctt

SEQ ID NO: 157 - DNA, 55 bp, Artificial
  synthetically derived forward primer
ggagcacagagatggaacagaatcggcacgcttcagaccggccctgcgtcatctc

SEQ ID NO: 158 - DNA, 57 bp, Artificial
  synthetically derived forward primer
ggagcaaccaagcacttgtcaatcggcacgcttcagacaggaaggccctggaaatcc

SEQ ID NO: 159 - DNA, 57 bp, Artificial
  synthetically derived forward primer
ggagcaacccgtcacttgtagatcggcacgcttcagacggatctgcgccgtttctgt

SEQ ID NO: 160 - DNA, 59 bp, Artificial
  synthetically derived forward primer
ggagcacaaacttccaaccgtatcggcacgcttcagactcctcgtgcgagaaagtgaga

SEQ ID NO: 161 - DNA, 59 bp, Artificial
  synthetically derived forward primer
ggagcacaagtttcgaacctgatcggcacgcttcagacgcatcatgatggccaattctg

SEQ ID NO: 162 - DNA, 61 bp, Artificial
  synthetically derived forward primer
ggagcaaacgagaacttgcccatcggcacgcttcagacgacgattctccaccgagtatgag

SEQ ID NO: 163 - DNA, 62 bp, Artificial
  synthetically derived forward primer
ggagcaacgcacagtaactacatcggcacgcttcagaccacggtggagatagtgacaatgtc

SEQ ID NO: 164 - DNA, 61 bp, Artificial
  synthetically derived forward primer
ggagcacaacgctacttgacaatcggcacgcttcagactacgggattccattcattgaaac

SEQ ID NO: 165 - DNA, 53 bp, Artificial
  synthetically derived forward primer
ggagcaaacgcctgtaagaagatcggcacgcttcagaccctgcgcgcctacct

SEQ ID NO: 166 - DNA, 57 bp, Artificial
  synthetically derived forward primer
ggagcaccaccctaattcttcatcggcacgcttcagactgcccaatgctgaccttca

SEQ ID NO: 167 - DNA, 60 bp, Artificial
  synthetically derived forward primer
ggagcacactttgcattctctatcggcacgcttcagactgccaagcttctctgtgaaaga

SEQ ID NO: 168 - DNA, 61 bp, Artificial
  synthetically derived forward primer
ggagcaacgtacatcaagaggatcggcacgcttcagactgatggaaaaggagttggacttg

SEQ ID NO: 169 - DNA, 61 bp, Artificial
  synthetically derived forward primer
ggagcacccacatccttctagatcggcacgcttcagacagaaccaagacccagacatcaag

SEQ ID NO: 170 - DNA, 56 bp, Artificial
  synthetically derived forward primer
ggagcacgatccacattctacatcggcacgcttcagacgccttgccccatcaactg

SEQ ID NO: 171 - DNA, 54 bp, Artificial
  synthetically derived forward primer
ggagcacgacctcgattcgtaatcggcacgcttcagacgcgcggcgatcaagaa

SEQ ID NO: 172 - DNA, 58 bp, Artificial
  synthetically derived forward primer
ggagcaagcctctctaagctgatcggcacgcttcagactcggtggtacctgagatgga

SEQ ID NO: 173 - DNA, 67 bp, Artificial
  synthetically derived forward primer
ggagcacaaaaacggttcgacatcggcacgcttcagaccaccctgatgaaaagattcaatgaaaact

SEQ ID NO: 174 - DNA, 59 bp, Artificial
  synthetically derived forward primer
ggagcacgatgatacttccggatcggcacgcttcagacgctcacccttccagacttcct

SEQ ID NO: 175 - DNA, 54 bp, Artificial
  synthetically derived forward primer
ggagcaacaaggtttaaggctatcggcacgcttcagacacgccatgcccattgg

SEQ ID NO: 176 - DNA, 63 bp, Artificial
  synthetically derived forward primer
ggagcaacctacgagttccaaatcggcacgcttcagacaaacctataggctcaggagctcaag

SEQ ID NO: 177 - DNA, 57 bp, Artificial
  synthetically derived forward primer
ggagcacgactaacatgttgaatcggcacgcttcagacctggagcggccaagtaggt

SEQ ID NO: 178 - DNA, 59 bp, Artificial
  synthetically derived forward primer
ggagcacacactggttgttcgatcggcacgcttcagacacccgctgtatggaaggaaac

SEQ ID NO: 179 - DNA, 61 bp, Artificial
  synthetically derived forward primer
ggagcaaacgagtcctgttccatcggcacgcttcagacggtagatggaaaaccggtgaatc

SEQ ID NO: 180 - DNA, 59 bp, Artificial
  synthetically derived forward primer
ggagcaccgtttctttgtgtgatcggcacgcttcagaccgagtccaaggcacatatcca

SEQ ID NO: 181 - DNA, 60 bp, Artificial
  synthetically derived forward primer
ggagcaaagcacgtctgtgtcatcggcacgcttcagacctgtgactcctccccagtatcc

SEQ ID NO: 182 - DNA, 61 bp, Artificial
  synthetically derived forward primer
ggagcaacagttgacacacggatcggcacgcttcagaccaattggagaaggacagcaacac

SEQ ID NO: 183 - DNA, 62 bp, Artificial
  synthetically derived forward primer
ggagcaccagataagacacgtatcggcacgcttcagactggtcaggcagtataatccaaaga

SEQ ID NO: 184 - DNA, 58 bp, Artificial
  synthetically derived forward primer
ggagcacaggataccacaggaatcggcacgcttcagacctggtggctgacgacaatca

SEQ ID NO: 185 - DNA, 61 bp, Artificial
  synthetically derived forward primer
ggagcaacactatacacaggcatcggcacgcttcagacggttatcaactcagtggcctttg

SEQ ID NO: 186 - DNA, 62 bp, Artificial
  synthetically derived forward primer
ggagcaaaacgtgaaacatcgatcggcacgcttcagacgcagtcaacagctaaaaccttcct

SEQ ID NO: 187 - DNA, 57 bp, Artificial
  synthetically derived forward primer
ggagcaagcatcctcacatctatcggcacgcttcagacacctggccgaggacatcat

SEQ ID NO: 188 - DNA, 60 bp, Artificial
  synthetically derived forward primer
ggagcaccgtgcatttgtaagatcggcacgcttcagactcccatcatctatgccttcgtt

SEQ ID NO: 189 - DNA, 63 bp, Artificial
  synthetically derived forward primer
ggagcaccacccttctgtaacatcggcacgcttcagactgacagtggcagctatgaacaaaat

SEQ ID NO: 190 - DNA, 55 bp, Artificial
  synthetically derived forward primer
ggagcaaaaagcgcatggttgatcggcacgcttcagacaagcaggctggcggaaa

SEQ ID NO: 191 - DNA, 59 bp, Artificial
  synthetically derived forward primer
ggagcacggatattcaccactatcggcacgcttcagacctacctcactggctgggatga

SEQ ID NO: 192 - DNA, 60 bp, Artificial
  synthetically derived forward primer
ggagcaaacaagaactggtctatcggcacgcttcagactgaacttccctggtcgaacagt

SEQ ID NO: 193 - DNA, 55 bp, Artificial
  synthetically derived forward primer
ggagcaccatcaaagaccattatcggcacgcttcagactcggagccctggaagca

SEQ ID NO: 194 - DNA, 58 bp, Artificial
  synthetically derived forward primer
ggagcaagaggcgtctggtaaatcggcacgcttcagacagtttgcccgggagagactt

SEQ ID NO: 195 - DNA, 60 bp, Artificial
  synthetically derived forward primer
ggagcaaggagactatgggtcatcggcacgcttcagacctggcaactctgaagtcatcct

SEQ ID NO: 196 - DNA, 62 bp, Artificial
  synthetically derived forward primer
ggagcaaaagctctctgggagatcggcacgcttcagaccaagacaattgtcaccaggatcaa

SEQ ID NO: 197 - DNA, 62 bp, Artificial
  synthetically derived forward primer
ggagcacaattgtgatgggaaatcggcacgcttcagacaaaagaatgtccatggtggtgtct

SEQ ID NO: 198 - DNA, 63 bp, Artificial
  synthetically derived forward primer
ggagcacactaacattggctcatcggcacgcttcagacccacattcagcaagtaggaaaattt

SEQ ID NO: 199 - DNA, 21 bp, Artificial
  synthetically derived universal forward primer
ggagcacaccattagttgtgg

SEQ ID NO: 200 - DNA, 21 bp, Artificial
  synthetically derived forward primer
ggagcaaaacaggacaacacg

SEQ ID NO: 201 - DNA, 21 bp, Artificial
  synthetically derived forward primer
ggagcacagagatggaacaga

SEQ ID NO: 202 - DNA, 21 bp, Artificial
  synthetically derived forward primer
ggagcaaccaagcacttgtca

SEQ ID NO: 203 - DNA, 21 bp, Artificial
  synthetically derived forward primer
ggagcaacccgtcacttgtag

SEQ ID NO: 204 - DNA, 21 bp, Artificial
  synthetically derived forward primer
ggagcacaaacttccaaccgt

SEQ ID NO: 205 - DNA, 21 bp, Artificial
  synthetically derived forward primer
ggagcacaagtttcgaacctg

SEQ ID NO: 206 - DNA, 21 bp, Artificial
  synthetically derived forward primer
ggagcaaacgagaacttgccc

SEQ ID NO: 207 - DNA, 21 bp, Artificial
  synthetically derived forward primer
ggagcaacgcacagtaactac

SEQ ID NO: 208 - DNA, 21 bp, Artificial
  synthetically derived forward primer
ggagcacaacgctacttgaca

SEQ ID NO: 209 - DNA, 21 bp, Artificial
  synthetically derived forward primer
ggagcaaacgcctgtaagaag

SEQ ID NO: 210 - DNA, 21 bp, Artificial
  synthetically derived forward primer
ggagcaccaccctaattcttc

SEQ ID NO: 211 - DNA, 21 bp, Artificial
  synthetically derived forward primer
ggagcacactttgcattctct

SEQ ID NO: 212 - DNA, 21 bp, Artificial
  synthetically derived forward primer
ggagcaacgtacatcaagagg

SEQ ID NO: 213 - DNA, 21 bp, Artificial
  synthetically derived forward primer
ggagcacccacatccttctag

SEQ ID NO: 214 - DNA, 21 bp, Artificial
  synthetically derived forward primer
ggagcacgatccacattctac

SEQ ID NO: 215 - DNA, 21 bp, Artificial
  synthetically derived forward primer
ggagcacgacctcgattcgta

SEQ ID NO: 216 - DNA, 21 bp, Artificial
  synthetically derived forward primer
ggagcaagcctctctaagctg

SEQ ID NO: 217 - DNA, 21 bp, Artificial
  synthetically derived forward primer
ggagcacaaaaacggttcgac

SEQ ID NO: 218 - DNA, 21 bp, Artificial
  synthetically derived forward primer
ggagcacgatgatacttccgg

SEQ ID NO: 219 - DNA, 21 bp, Artificial
  synthetically derived forward primer
ggagcaacaaggtttaaggct

SEQ ID NO: 220 - DNA, 21 bp, Artificial
  synthetically derived forward primer
ggagcaacctacgagttccaa

SEQ ID NO: 221 - DNA, 21 bp, Artificial
  synthetically derived forward primer
ggagcacgactaacatgttga

SEQ ID NO: 222 - DNA, 21 bp, Artificial
  synthetically derived forward primer
ggagcacacactggttgttcg

SEQ ID NO: 223 - DNA, 21 bp, Artificial
  synthetically derived forward primer
ggagcaaacgagtcctgttcc

SEQ ID NO: 224 - DNA, 21 bp, Artificial
  synthetically derived forward primer
ggagcaccgtttctttgtgtg

SEQ ID NO: 225 - DNA, 21 bp, Artificial
  synthetically derived forward primer
ggagcaaagcacgtctgtgtc

SEQ ID NO: 226 - DNA, 21 bp, Artificial
  synthetically derived forward primer
ggagcaacagttgacacacgg

SEQ ID NO: 227 - DNA, 21 bp, Artificial
  synthetically derived forward primer
ggagcaccagataagacacgt

SEQ ID NO: 228 - DNA, 21 bp, Artificial
  synthetically derived forward primer
ggagcacaggataccacagga

SEQ ID NO: 229 - DNA, 21 bp, Artificial
  synthetically derived forward primer
ggagcaacactatacacaggc

SEQ ID NO: 230 - DNA, 21 bp, Artificial
  synthetically derived forward primer
ggagcaaaacgtgaaacatcg

SEQ ID NO: 231 - DNA, 21 bp, Artificial
  synthetically derived forward primer
ggagcaagcatcctcacatct

SEQ ID NO: 232 - DNA, 21 bp, Artificial
  synthetically derived forward primer
ggagcaccgtgcatttgtaag

SEQ ID NO: 233 - DNA, 21 bp, Artificial
  synthetically derived forward primer
ggagcaccacccttctgtaac

SEQ ID NO: 234 - DNA, 21 bp, Artificial
  synthetically derived forward primer
ggagcaaaaagcgcatggttg

SEQ ID NO: 235 - DNA, 21 bp, Artificial
  synthetically derived forward primer
ggagcacggatattcaccact

SEQ ID NO: 236 - DNA, 21 bp, Artificial
  synthetically derived forward primer
ggagcaaacaagaactggtct

SEQ ID NO: 237 - DNA, 21 bp, Artificial
  synthetically derived forward primer
ggagcaccatcaaagaccatt

SEQ ID NO: 238 - DNA, 21 bp, Artificial
  synthetically derived forward primer
ggagcaagaggcgtctggtaa

SEQ ID NO: 239 - DNA, 21 bp, Artificial
  synthetically derived forward primer
ggagcaaggagactatgggtc

SEQ ID NO: 240 - DNA, 21 bp, Artificial
  synthetically derived forward primer
ggagcaaaagctctctgggag

SEQ ID NO: 241 - DNA, 21 bp, Artificial
  synthetically derived forward primer
ggagcacaattgtgatgggaa

SEQ ID NO: 242 - DNA, 21 bp, Artificial
  synthetically derived forward primer
ggagcacactaacattggctc

SEQ ID NO: 243 - DNA, 17 bp, Artificial
  snythetically derived detection probe sequence
atcggcacgcttcagac

SEQ ID NO: 244 - DNA, 17 bp, Artificial
  snythetically derived detection probe sequence
atcggcacgcttcagac

SEQ ID NO: 245 - DNA, 17 bp, Artificial
  snythetically derived detection probe sequence
atcggcacgcttcagac

SEQ ID NO: 246 - DNA, 17 bp, Artificial
  snythetically derived detection probe sequence
atcggcacgcttcagac

SEQ ID NO: 247 - DNA, 17 bp, Artificial
  snythetically derived detection probe sequence
atcggcacgcttcagac

SEQ ID NO: 248 - DNA, 17 bp, Artificial
  snythetically derived detection probe sequence
atcggcacgcttcagac

SEQ ID NO: 249 - DNA, 17 bp, Artificial
  snythetically derived detection probe sequence
atcggcacgcttcagac

SEQ ID NO: 250 - DNA, 17 bp, Artificial
  snythetically derived detection probe sequence
atcggcacgcttcagac

SEQ ID NO: 251 - DNA, 17 bp, Artificial
  snythetically derived detection probe sequence
atcggcacgcttcagac

SEQ ID NO: 252 - DNA, 17 bp, Artificial
  snythetically derived detection probe sequence
atcggcacgcttcagac

SEQ ID NO: 253 - DNA, 17 bp, Artificial
  snythetically derived detection probe sequence
atcggcacgcttcagac

SEQ ID NO: 254 - DNA, 17 bp, Artificial
  snythetically derived detection probe sequence
atcggcacgcttcagac

SEQ ID NO: 255 - DNA, 17 bp, Artificial
  snythetically derived detection probe sequence
atcggcacgcttcagac

SEQ ID NO: 256 - DNA, 17 bp, Artificial
  snythetically derived detection probe sequence
atcggcacgcttcagac

SEQ ID NO: 257 - DNA, 17 bp, Artificial
  snythetically derived detection probe sequence
atcggcacgcttcagac

SEQ ID NO: 258 - DNA, 17 bp, Artificial
  snythetically derived detection probe sequence
atcggcacgcttcagac

SEQ ID NO: 259 - DNA, 17 bp, Artificial
  snythetically derived detection probe sequence
atcggcacgcttcagac

SEQ ID NO: 260 - DNA, 17 bp, Artificial
  snythetically derived detection probe sequence
atcggcacgcttcagac

SEQ ID NO: 261 - DNA, 17 bp, Artificial
  snythetically derived detection probe sequence
atcggcacgcttcagac

SEQ ID NO: 262 - DNA, 17 bp, Artificial
  snythetically derived detection probe sequence
atcggcacgcttcagac

SEQ ID NO: 263 - DNA, 17 bp, Artificial
  snythetically derived detection probe sequence
atcggcacgcttcagac

SEQ ID NO: 264 - DNA, 17 bp, Artificial
  snythetically derived detection probe sequence
atcggcacgcttcagac

SEQ ID NO: 265 - DNA, 17 bp, Artificial
  snythetically derived detection probe sequence
atcggcacgcttcagac

SEQ ID NO: 266 - DNA, 17 bp, Artificial
  snythetically derived detection probe sequence
atcggcacgcttcagac

SEQ ID NO: 267 - DNA, 17 bp, Artificial
  snythetically derived detection probe sequence
atcggcacgcttcagac

SEQ ID NO: 268 - DNA, 17 bp, Artificial
  snythetically derived detection probe sequence
atcggcacgcttcagac

SEQ ID NO: 269 - DNA, 17 bp, Artificial
  snythetically derived detection probe sequence
atcggcacgcttcagac

SEQ ID NO: 270 - DNA, 17 bp, Artificial
  snythetically derived detection probe sequence
atcggcacgcttcagac

SEQ ID NO: 271 - DNA, 17 bp, Artificial
  snythetically derived detection probe sequence
atcggcacgcttcagac

SEQ ID NO: 272 - DNA, 17 bp, Artificial
  snythetically derived detection probe sequence
atcggcacgcttcagac

SEQ ID NO: 273 - DNA, 17 bp, Artificial
  snythetically derived detection probe sequence
atcggcacgcttcagac

SEQ ID NO: 274 - DNA, 17 bp, Artificial
  snythetically derived detection probe sequence
atcggcacgcttcagac

SEQ ID NO: 275 - DNA, 17 bp, Artificial
  snythetically derived detection probe sequence
atcggcacgcttcagac

SEQ ID NO: 276 - DNA, 17 bp, Artificial
  snythetically derived detection probe sequence
atcggcacgcttcagac

SEQ ID NO: 277 - DNA, 17 bp, Artificial
  snythetically derived detection probe sequence
atcggcacgcttcagac

SEQ ID NO: 278 - DNA, 17 bp, Artificial
  snythetically derived detection probe sequence
atcggcacgcttcagac

SEQ ID NO: 279 - DNA, 17 bp, Artificial
  snythetically derived detection probe sequence
atcggcacgcttcagac

SEQ ID NO: 280 - DNA, 17 bp, Artificial
  snythetically derived detection probe sequence
atcggcacgcttcagac

SEQ ID NO: 281 - DNA, 17 bp, Artificial
  snythetically derived detection probe sequence
atcggcacgcttcagac

SEQ ID NO: 282 - DNA, 17 bp, Artificial
  snythetically derived detection probe sequence
atcggcacgcttcagac

SEQ ID NO: 283 - DNA, 17 bp, Artificial
  snythetically derived detection probe sequence
atcggcacgcttcagac

SEQ ID NO: 284 - DNA, 17 bp, Artificial
  snythetically derived detection probe sequence
atcggcacgcttcagac

SEQ ID NO: 285 - DNA, 17 bp, Artificial
  snythetically derived detection probe sequence
atcggcacgcttcagac

SEQ ID NO: 286 - DNA, 17 bp, Artificial
  snythetically derived detection probe sequence
atcggcacgcttcagac

SEQ ID NO: 287 - DNA, 23 bp, Artificial
  synthetically derived target sequence
accgtgtgaatcattgtctgaca

SEQ ID NO: 288 - DNA, 21 bp, Artificial
  synthetically derived target sequence
gtgtggtctggtgcctgtctt

SEQ ID NO: 289 - DNA, 17 bp, Artificial
  synthetically derived target sequence
cggccctgcgtcatctc

SEQ ID NO: 290 - DNA, 19 bp, Artificial
  synthetically derived target sequence
aggaaggccctggaaatcc

SEQ ID NO: 291 - DNA, 19 bp, Artificial
  synthetically derived target sequence
ggatctgcgccgtttctgt

SEQ ID NO: 292 - DNA, 21 bp, Artificial
  synthetically derived target sequence
tcctcgtgcgagaaagtgaga

SEQ ID NO: 293 - DNA, 21 bp, Artificial
  synthetically derived target sequence
gcatcatgatggccaattctg

SEQ ID NO: 294 - DNA, 23 bp, Artificial
  synthetically derived target sequence
gacgattctccaccgagtatgag

SEQ ID NO: 295 - DNA, 24 bp, Artificial
  synthetically derived target sequence
cacggtggagatagtgacaatgtc

SEQ ID NO: 296 - DNA, 23 bp, Artificial
  synthetically derived target sequence
tacgggattccattcattgaaac

SEQ ID NO: 297 - DNA, 15 bp, Artificial
  synthetically derived target sequence
cctgcgcgcctacct

SEQ ID NO: 298 - DNA, 19 bp, Artificial
  synthetically derived target sequence
tgcccaatgctgaccttca

SEQ ID NO: 299 - DNA, 22 bp, Artificial
  synthetically derived target sequence
tgccaagcttctctgtgaaaga

SEQ ID NO: 300 - DNA, 23 bp, Artificial
  synthetically derived target sequence
tgatggaaaaggagttggacttg

SEQ ID NO: 301 - DNA, 23 bp, Artificial
  synthetically derived target sequence
agaaccaagacccagacatcaag

SEQ ID NO: 302 - DNA, 18 bp, Artificial
  synthetically derived target sequence
gccttgccccatcaactg

SEQ ID NO: 303 - DNA, 16 bp, Artificial
  synthetically derived target sequence
gcgcggcgatcaagaa

SEQ ID NO: 304 - DNA, 20 bp, Artificial
  synthetically derived target sequence
tcggtggtacctgagatgga

SEQ ID NO: 305 - DNA, 29 bp, Artificial
  synthetically derived target sequence
caccctgatgaaaagattcaatgaaaact

SEQ ID NO: 306 - DNA, 21 bp, Artificial
  synthetically derived target sequence
gctcacccttccagacttcct

SEQ ID NO: 307 - DNA, 16 bp, Artificial
  synthetically derived target sequence
acgccatgcccattgg

SEQ ID NO: 308 - DNA, 25 bp, Artificial
  synthetically derived target sequence
aaacctataggctcaggagctcaag

SEQ ID NO: 309 - DNA, 21 bp, Artificial
  synthetically derived target sequence
acccgctgtatggaaggaaac

SEQ ID NO: 310 - DNA, 21 bp, Artificial
  synthetically derived target sequence
acccgctgtatggaaggaaac

SEQ ID NO: 311 - DNA, 23 bp, Artificial
  synthetically derived target sequence
ggtagatggaaaaccggtgaatc

SEQ ID NO: 312 - DNA, 21 bp, Artificial
  synthetically derived target sequence
cgagtccaaggcacatatcca

SEQ ID NO: 313 - DNA, 22 bp, Artificial
  synthetically derived target sequence
ctgtgactcctccccagtatcc

SEQ ID NO: 314 - DNA, 23 bp, Artificial
  synthetically derived target sequence
caattggagaaggacagcaacac

SEQ ID NO: 315 - DNA, 24 bp, Artificial
  synthetically derived target sequence
tggtcaggcagtataatccaaaga

SEQ ID NO: 316 - DNA, 20 bp, Artificial
  synthetically derived target sequence
ctggtggctgacgacaatca

SEQ ID NO: 317 - DNA, 20 bp, Artificial
  synthetically derived target sequence
ctggtggctgacgacaatca

SEQ ID NO: 318 - DNA, 24 bp, Artificial
  synthetically derived target sequence
gcagtcaacagctaaaaccttcct

SEQ ID NO: 319 - DNA, 19 bp, Artificial
  synthetically derived target sequence
acctggccgaggacatcat

SEQ ID NO: 320 - DNA, 22 bp, Artificial
  synthetically derived target sequence
tcccatcatctatgccttcgtt

SEQ ID NO: 321 - DNA, 25 bp, Artificial
  synthetically derived target sequence
tgacagtggcagctatgaacaaaat

SEQ ID NO: 322 - DNA, 17 bp, Artificial
  synthetically derived target sequence
aagcaggctggcggaaa

SEQ ID NO: 323 - DNA, 21 bp, Artificial
  synthetically derived target sequence
ctacctcactggctgggatga

SEQ ID NO: 324 - DNA, 22 bp, Artificial
  synthetically derived target sequence
tgaacttccctggtcgaacagt

SEQ ID NO: 325 - DNA, 17 bp, Artificial
  synthetically derived target sequence
tcggagccctggaagca

SEQ ID NO: 326 - DNA, 20 bp, Artificial
  synthetically derived target sequence
agtttgcccgggagagactt

SEQ ID NO: 327 - DNA, 22 bp, Artificial
  synthetically derived target sequence
ctggcaactctgaagtcatcct

SEQ ID NO: 328 - DNA, 24 bp, Artificial
  synthetically derived target sequence
caagacaattgtcaccaggatcaa

SEQ ID NO: 329 - DNA, 24 bp, Artificial
  synthetically derived target sequence
aaaagaatgtccatggtggtgtct

SEQ ID NO: 330 - DNA, 25 bp, Artificial
  synthetically derived target sequence
ccacattcagcaagtaggaaaattt